(19) United States
(12) Patent Application Publication
(10) Pub. No.: US 2021/0231628 A1
(43) Pub. Date: Jul. 29, 2021

(54) PHOTOLUMINESCENCE-BASED DETECTION OF ACID GASES VIA RARE EARTH METAL-ORGANIC FRAMEWORKS

(71) Applicant: National Technology & Engineering Solutions of Sandia, LLC, Albuquerque, NM (US)

(72) Inventors: Tina M. Nenoff, Albuquerque, NM (US); Dorina F. Sava Gallis, Albuquerque, NM (US); Dayton Jonathan Vogel, Albuquerque, NM (US); Jessica Rimsza, Albuquerque, NM (US)

(21) Appl. No.: 17/142,443

(22) Filed: Jan. 6, 2021

Related U.S. Application Data
(60) Provisional application No. 62/958,184, filed on Jan. 7, 2020.

(51) Int. Cl. G01N 33/00; C07F 5/00; G01N 21/64

(52) U.S. Cl. CPC G01N 33/0037; G01N 21/64; C07F 5/00

(57) ABSTRACT

The present invention relates to a metal-organic framework composition, as well as constructs and methods thereof. In one particular example, the composition is employed to detect the presence of an acid gas.

### CROSS-REFERENCE TO RELATED APPLICATION

This application claims the benefit of U.S. Provisional Application No. 62/958,184, filed Jan. 7, 2020, which is incorporated herein by reference.

### STATEMENT OF GOVERNMENT INTEREST

This invention was made with Government support under Contract No. DE-NA0003525 awarded by the United States Department of Energy/National Nuclear Security Administration. The Government has certain rights in the invention.

### FIELD OF THE INVENTION

The present invention relates to a metal-organic framework composition, as well as constructs and methods thereof. In one particular example, the composition is employed to detect the presence of an acid gas.

### BACKGROUND OF THE INVENTION

Acid gases are found throughout energy applications and are common in flue gas treatments, which often contain parts per million levels of NOₓ (e.g., NO₂/NO), SOₓ, and humidity (H₂O). See F. Rezaei et al., Energy Fuels 29, 5467 (2015); F. Normann et al., Int. J. Greenhouse Gas Control 12, 26 (2013); F. Rezaei et al., Ind. Eng. Chem. Res. 52, 12192 (2013); and K. S. Walton et al., Joule 1, 208 (2017). Toxicity of flue gas streams is an important environmental concern since ozone forms when NOₓ species interact with other airborne volatile organic compounds. In particular, such acid gases are commonly found in complex chemical and petrochemical streams and require material development for their selective adsorption and removal. Currently, removal of NOₓ in industrial settings is done through an energy intensive catalyst driven process. See F. Rezaei et al., Energy Fuels 29, 5467 (2015); and M. Shelef, Chem. Rev. 95, 209 (1995). To reduce the complexity and limit the energy penalties of existing processing conditions, adsorption by conventional porous supports has been investigated. See A. K. Das et al., AIChE J. 47, 2831 (2001); P. Davini, Carbon 39, 2173 (2001); H. Deng et al., Chem. Eng. J. 188, 77 (2012); and W. Sun et al., AIChE J. 60, 2314 (2014).

Therefore, metal-organic frameworks (MOFs) have emerged as attractive candidates for gas adsorption and separation. See D. F. Sava Gallis et al., Chem. Mater. 28, 3327 (2016); M. V. Parkes et al., Phys. Chem. Chem. Phys. 18, 11528 (2016); M. V. Parkes et al., J. Phys. Chem. C 119, 6556 (2015); D. F. Sava Gallis et al., Chem. Mater. 27, 2018 (2015); S. Ma et al., Chem. Commun. 46, 44 (2010); J. R. Li et al., Chem. Soc. Rev. 38, 1477 (2009); J. R. Li et al., Chem. Rev. 112, 869 (2012); Y. He et al., Chem. Soc. Rev. 43, 5657 (2014); and O. K. Farha et al., Acc. Chem. Res. 43, 1166 (2010). MOFs possess unique attributes that are highly advantageous for gas adsorption, including increased surface area, pore sizes, pore volumes, and chemical tunability. MOF materials have been extensively investigated for CO₂ removal from flue gas mixed streams, including several recent studies for NOx removal applications. See G. Férey et al., Chem. Soc. Rev. 40, 550 (2011); J. Liu et al., Chem. Soc. Rev. 41, 2308 (2012); S. Han et al., ACS Comb. Sci. 14, 263 (2012); A. M. Ebrahim et al., Langmuir 29, 168 (2013); A. M. Ebrahim et al., Microporous Mesoporous Mater. 188, 149 (2014); J. B. DeCoste et al., New J. Chem. 39, 2396 (2015); G. W. Peterson et al., Angew. Chem. Int. Ed. 55, 6235 (2016); and K. Tan et al., Chem. Mater. 29, 4227 (2017). In particular, zeolitic imidazolate frameworks (ZIFs) and Zr-based MOF materials have been shown to resist degradation when exposed to corrosive environments (NOₓ and SOₓ), with varying stability in dry and humid conditions. See K. S. Park et al., Proc. Nat'l Acad. Sci. USA 103, 10186 (2006); S. Bhattacharyya et al., Chem. Mater. 30, 4089 (2018); and S. Bhattacharyya et al., J. Phys. Chem. C 123, 2336 (2019).

When MOFs have been unstable to acid gases, the breakdown has occurred mainly on the organic ligand. See S. Bhattacharyya et al., Chem. Mater. 30, 4089 (2018). Therefore, in the design of MOFs durable to acid gases, it is important to consider the acid gas binding affinity to the metal center, especially in the context of complex gas mixtures. Interestingly, it has been shown that various lanthanide metals exhibit preferential binding to acid gases. For example, flue gas streams have been scrubbed of NOₓ and SOₓ by lanthanide oxygen-sulfur catalysts. See D. A. R. Kay et al., U.S. Pat. No. 5,213,779, issued May 25, 1993. And there are numerous examples of favorable lanthanide-H₂S binding. A recent study has demonstrated the efficacy of rare earth fcu-MOFs to selectively remove H₂S from CO₂-containing gas streams, as well as a strong selectivity and coordination binding to H₂S. See P. M. Bhatt et al., Chem. Eng. J 324, 392 (2017); B. Liu et al., Anal. Chem. 85, 11020 (2013); and Y. W. Yip et al., Dalton Trans. 45, 928 (2016).

However, detection and selective capture of such acid gases remain a technical challenge. In particular, there is a need for additional materials displaying affinity, durability, and selectivity for such gases.

### SUMMARY OF THE INVENTION

The present invention relates, in part, to methods and compositions for capturing and detecting one or more acid gases. In particular, the compositions includes a metal-organic framework (MOF), as described herein. Accordingly, in a first aspect, the present invention is directed to a method of detecting an acid gas, the method including: providing a metal-organic framework composition including a plurality of metal clusters and a plurality of ligands coordinating with the plurality of metal clusters; exposing the metal-organic framework composition to the acid gas; and detecting a change in an optical emission spectrum of the metal-organic framework composition, as compared to before exposure to the acid gas.

In a second aspect, the present invention features a method of capturing an acid gas, the method including: providing a metal-organic framework composition (e.g., any described herein, such as a composition including a plurality of metal clusters and a plurality of ligands coordinating with the plurality of metal clusters); and exposing the metal-organic framework composition to the acid gas.

In some embodiments, the method further includes detecting a change in an optical emission spectrum of the metal-organic framework composition, as compared to before exposure to the acid gas, thereby confirming capture of the acid gas.

In any embodiment herein, the optical emission spectrum is a photoluminescence spectrum, a photoluminescence excitation spectrum, and/or a photoluminescence emission spectrum. In other embodiments, the detecting step includes monitoring an emission spectrum while scanning an excitation spectrum of the metal-organic framework composition before and after exposure to the acid gas. In yet other embodiments, the detecting step includes exciting the metal-organic framework composition with an ultraviolet light and monitoring a decrease in an emission intensity at a wavelength within a visible spectrum, as compared to before exposure to the acid gas. In particular embodiments, the ultraviolet light has a wavelength of from about 320 to about 400 nm. In other embodiments, the visible spectrum has a range of from about 400 nm to about 650 nm.

In any embodiment herein, the acid gas can comprise a nitrogen oxide (i.e., NOₓ), sulfur oxide (i.e., SOₓ), hydrogen sulfide, and carbon dioxide.

In any embodiment herein, at least one of the plurality of metal clusters includes a hexanuclear cluster. In some embodiments, at least one of the plurality of metal clusters includes a metal ion, at least one of the plurality of ligands can be a monodentate ligand, and at least one of the plurality of ligands can be a bidentate ligand. In other embodiments, at least one hexanuclear cluster or all hexanuclear clusters include Zr, Eu, Nd, Yb, Y, Tb, La, Ce, Pr, Sm, Gd, Dy, Ho, Er, Tm, and/or Lu.

In any embodiment herein, the plurality of metal clusters includes a first metal ion and a second metal ion that is different than the first metal ion. In some embodiments, the plurality of metal clusters includes a first metal ion having a first coordination geometry and a second metal ion having a second coordination geometry that is different than the first coordinate geometry.

In any embodiment herein, the metal-organic framework composition includes a plurality of monodentate ligands and/or a plurality of bidentate ligands. In some embodiments, at least one of the plurality of ligands includes a structure of L¹-R^{L}-L², wherein each of L¹ and L² is, independently, a reactive group (e.g., any described herein), and wherein R^{L} is a linker (e.g., any described herein). In other embodiments, R^{L} includes an optionally substituted aryl or an optionally substituted heteroaryl (e.g., an aryl substituted with one or more of a hydroxyl, optionally substituted alkyl, haloalkyl, hydroxyalkyl, optionally substituted alkoxy, optionally substituted cycloalkyl, optionally substituted cycloalkoxy, optionally substituted aryl, optionally substituted aryloxy, halo, carboxyl, azido, cyano, nitro, amino, aminoalkyl, or carboxyaldehyde). In some embodiments, each of L¹ and L² includes, independently, carboxyl, heterocyclyl, hydroxyl, an anion thereof, a salt thereof, or an ester thereof.

In any embodiment herein, the plurality of metal clusters and plurality of ligands form a periodic framework.

In any embodiment herein, at least one of the plurality of ligands comprises a linear dicarboxylic acid.

In any embodiment herein, the metal-organic framework composition includes EuDOBDC (DOBDC; 2,5-dihydroxyterephthalic acid or 2,5-dihydroxy-1,4-benzenedicarboxylic acid), YDOBDC, NdDOBDC, YbDOBDC, TbDOBDC, Nd_{0.67}Yb_{0.33}DOBDC, Nd_{0.46}Yb_{0.54}DOBDC, UiO-66-DOBDC, UiO-66, UiO-67, NU-1000, MOF-808, or PCN-777.

### Definitions

As used herein, the term “about” means+/−10% of any recited value. As used herein, this term modifies any recited value, range of values, or endpoints of one or more ranges.

By “alkoxy” is meant —OR, where R is an optionally substituted alkyl group, as described herein. Exemplary alkoxy groups include methoxy, ethoxy, butoxy, trihaloalkoxy, such as trifluoromethoxy, etc. The alkoxy group can be substituted or unsubstituted. For example, the alkoxy group can be substituted with one or more substitution groups, as described herein for alkyl. Exemplary unsubstituted alkoxy groups include C₁₋₃, C₁₋₆, C₁₋₁₂, C₁₋₁₆, C₁₋₁₈, C₁₋₂₀, or C₁₋₂₄ alkoxy groups.

By “alkyl” and the prefix “alk” is meant a branched or unbranched saturated hydrocarbon group of 1 to 24 carbon atoms, such as methyl, ethyl, n-propyl, isopropyl, n-butyl, isobutyl, s-butyl, t-butyl, n-pentyl, isopentyl, s-pentyl, neopentyl, hexyl, heptyl, octyl, nonyl, decyl, dodecyl, tetradecyl, hexadecyl, eicosyl, tetracosyl, and the like. The alkyl group can be cyclic (e.g., C₃₋₂₄ cycloalkyl) or acyclic. The alkyl group can be branched or unbranched. The alkyl group can also be substituted or unsubstituted. For example, the alkyl group can be substituted with one, two, three or, in the case of alkyl groups of two carbons or more, four substituents independently selected from the group consisting of: (1) C₁₋₆ alkoxy (e.g., —OAk, in which Ak is an alkyl group, as defined herein); (2) C₁₋₆ alkylsulfinyl (e.g., —S(O)Ak, in which Ak is an alkyl group, as defined herein); (3) C₁₋₆ alkylsulfonyl (e.g., —SO₂Ak, in which Ak is an alkyl group, as defined herein); (4) amino (e.g., —NR^{N1}R^{N2}, where each of R^{N1} and R^{N2} is, independently, H or optionally substituted alkyl, or R^{N1} and R^{N2}, taken together with the nitrogen atom to which each are attached, form a heterocyclyl group); (5) aryl; (6) arylalkoxy (e.g., —OA^{L}Ar, in which A^{L} is an alkylene group and Ar is an aryl group, as defined herein); (7) aryloyl (e.g., —C(O)Ar, in which Ar is an aryl group, as defined herein); (8) azido (e.g., an —N₃ group); (9) cyano (e.g., a —CN group); (10) carboxyaldehyde (e.g., a —C(O)H group); (11) C₃— cycloalkyl; (12) halo; (13) heterocyclyl (e.g., a 5-, 6- or 7-membered ring, unless otherwise specified, containing one, two, three, or four non-carbon heteroatoms (e.g., independently selected from the group consisting of nitrogen, oxygen, phosphorous, sulfur, or halo)); (14) heterocyclyloxy (e.g., —OHet, in which Het is a heterocyclyl group); (15) heterocyclyloyl (e.g., —C(O)Het, in which Het is a heterocyclyl group); (16) hydroxyl (e.g., a —OH group); (17)N-protected amino; (18) nitro (e.g., an —NO₂ group); (19) oxo (e.g., an ═O group); (20) C₃₋₈ spirocyclyl (e.g., an alkylene diradical, both ends of which are bonded to the same carbon atom of the parent group to form a spirocyclyl group); (21) C₁₋₆ thioalkoxy (e.g., —SAk, in which Ak is an alkyl group, as defined herein); (22) thiol (e.g., an —SH group); (23) —CO₂R^{A}, where R^{A} is selected from the group consisting of (a) hydrogen, (b) C₁₋₆ alkyl, (c) C₄₋₁₈ aryl, and (d) C₁₋₆ alk-C₄₋₁₈ aryl; (24) —C(O)NR^{B}R^{C}, where each of R^{B} and R^{C} is, independently, selected from the group consisting of (a) hydrogen, (b) C₁₋₆ alkyl, (c) C₄₋₁₈ aryl, and (d) C₁₋₆ alk-C₄₋₁₈ aryl; (25) —SO₂R^{D}, where R^{D} is selected from the group consisting of (a) C₁₋₆ alkyl, (b) C₄₋₁₈ aryl, and (c) C₁₋₆ alk-C₄₋₁₈ aryl; (26) —SO₂NR^{E}R^{F}, where each of R^{E} and R^{F} is, independently, selected from the group consisting of (a) hydrogen, (b) C₁₋₆ alkyl, (c) C₄₋₁₈ aryl, and (d) C₁₋₆ alk-C₄₋₁₈ aryl; and (27) —NR^{G}R^{H}, where each of R^{G} and R^{H} is, independently, selected from the group consisting of (a) hydrogen, (b) an N-protecting group, (c) C₁₋₆ alkyl, (d) C₂₋₆ alkenyl, (e) C₂₋₆ alkynyl, (f) C₄₋₁₈ aryl, (g) C₁₋₆ alk-C₄₋₁₈ aryl, (h) C₃-cycloalkyl, and (i) C₁₋₆ alk-C₃₋₈ cycloalkyl, wherein in one embodiment no two groups are bound to the nitrogen atom through a carbonyl group or a sulfonyl group. The alkyl group can be a primary, secondary, or tertiary alkyl group substituted with one or more substituents (e.g., one or more halo or alkoxy). In some embodiments, the unsubstituted alkyl group is a C₁₋₃, C₁₋₆, C₁₋₁₂, C₁₋₁₆, C₁₋₁₈, C₁₋₂₀, or C₁₋₂₄ alkyl group.

By “alkylene” is meant a multivalent (e.g., bivalent, trivalent, tetravalent, etc.) form of an alkyl group, as described herein. Exemplary alkylene groups include methylene, ethylene, propylene, butylene, etc. In some embodiments, the alkylene group is a C₁₋₃, C₁₋₆, C₁₋₁₂, C₁₋₁₆, C₁₋₁₈, C₁₋₂₀, C₁₋₂₄, C₂₋₃, C₂₋₆, C₂₋₁₂, C₂₋₁₆, C₂₋₁₈, C₂₋₂₀, or C₂₋₂₄ alkylene group. The alkylene group can be branched or unbranched. The alkylene group can also be substituted or unsubstituted. For example, the alkylene group can be substituted with one or more substitution groups, as described herein for alkyl.

By “amino” is meant —NR^{N1}R^{N2}, where each of R^{N1} and R^{N2} is, independently, H or optionally substituted alkyl, or R^{N1} and R^{N2}, taken together with the nitrogen atom to which each are attached, form a heterocyclyl group, as defined herein.

By “aminoalkyl” is meant an alkyl group, as defined herein, substituted by an amino group, as defined herein.

By “aryl” is meant a group that contains any carbon-based aromatic group including, but not limited to, benzyl, naphthalene, phenyl, biphenyl, phenoxybenzene, anthracene, and the like. The term “aryl” also includes “heteroaryl,” which is defined as a group that contains an aromatic group that has at least one heteroatom incorporated within the ring of the aromatic group. Examples of heteroatoms include, but are not limited to, nitrogen, oxygen, sulfur, and phosphorus. Likewise, the term “non-heteroaryl,” which is also included in the term “aryl,” defines a group that contains an aromatic group that does not contain a heteroatom. The aryl group can be substituted or unsubstituted. The aryl group can be substituted with one, two, three, four, or five substituents independently selected from the group consisting of: (1) C₁₋₆ alkanoyl (e.g., —C(O)Ak, in which Ak is an alkyl group, as defined herein); (2) C₁₋₆ alkyl; (3) C₁₋₆ alkoxy (e.g., —OAk, in which Ak is an alkyl group, as defined herein); (4) C₁₋₆ alkoxy-C₁₋₆ alkyl (e.g., an alkyl group, which is substituted with an alkoxy group —OAk, in which Ak is an alkyl group, as defined herein); (5) C₁₋₆ alkylsulfinyl (e.g., —S(O)Ak, in which Ak is an alkyl group, as defined herein); (6) C₁₋₆ alkylsulfinyl-C₁₋₆ alkyl (e.g., an alkyl group, which is substituted by an alkylsulfinyl group —S(O)Ak, in which Ak is an alkyl group, as defined herein); (7) C₁₋₆ alkylsulfonyl (e.g., —SO₂Ak, in which Ak is an alkyl group, as defined herein); (8) C₁₋₆ alkylsulfonyl-C₁₋₆ alkyl (e.g., an alkyl group, which is substituted by an alkylsulfonyl group —SO₂Ak, in which Ak is an alkyl group, as defined herein); (9) aryl; (10) amino (e.g., —NR^{N1}R^{N2} where each of R^{N1} and R^{N2} is, independently, H or optionally substituted alkyl, or R^{N1} and R^{N2}, taken together with the nitrogen atom to which each are attached, form a heterocyclyl group); (11) C₁₋₆ aminoalkyl (e.g., meant an alkyl group, as defined herein, substituted by an amino group); (12) heteroaryl; (13) C₁₋₆ alk-C₄₋₁₈ aryl (e.g., -A^{L}Ar, in which A^{L} is an alkylene group and Ar is an aryl group, as defined herein); (14) aryloyl (e.g., —C(O)Ar, in which Ar is an aryl group, as defined herein); (15) azido (e.g., an —N₃ group); (16) cyano (e.g., a —CN group); (17) C₁₋₆ azidoalkyl (e.g., a —N₃ azido group attached to the parent molecular group through an alkyl group, as defined herein); (18) carboxyaldehyde (e.g., a —C(O)H group); (19) carboxyaldehyde-C₁₋₆ alkyl (e.g., -A^{L}C(O)H, in which A^{L} is an alkylene group, as defined herein); (20) C₃₋₈ cycloalkyl; (21) C₁₋₆ alk-C₃₋₈ cycloalkyl (e.g., -A^{L}Cy, in which A^{L} is an alkylene group and Cy is a cycloalkyl group, as defined herein); (22) halo (e.g., F, Cl, Br, or I); (23) C₁₋₆ haloalkyl (e.g., an alkyl group, as defined herein, substituted with one or more halo); (24) heterocyclyl; (25) heterocyclyloxy (e.g., —OHet, in which Het is a heterocyclyl group); (26) heterocyclyloyl (e.g., —C(O)Het, in which Het is a heterocyclyl group); (16) hydroxyl (e.g., a —OH group); (27) hydroxyl (e.g., a —OH group); (28) C₁₋₆ hydroxyalkyl (e.g., an alkyl group, as defined herein, substituted by one to three hydroxyl groups, with the proviso that no more than one hydroxyl group may be attached to a single carbon atom of the alkyl group); (29) nitro (e.g., an —NO₂ group); (30) C₁₋₆ nitroalkyl (e.g., an alkyl group, as defined herein, substituted by one to three nitro groups); (31)N-protected amino; (32)N-protected amino-C₁₋₆ alkyl; (33) oxo (e.g., an ═O group); (34) C₁₋₆ thioalkoxy (e.g., -SAk, in which Ak is an alkyl group, as defined herein); (35) thio-C₁₋₆ alkoxy-C₁₋₆ alkyl (e.g., an alkyl group, which is substituted by an thioalkoxy group —SAk, in which Ak is an alkyl group, as defined herein); (36) —(CH₂)ᵣCO₂R^{A}, where r is an integer of from zero to four, and R^{A} is selected from the group consisting of (a) hydrogen, (b) C₁₋₆ alkyl, (c) C₄₋₁₈ aryl, and (d) C₁₋₆ alk-C₄₋₁₈ aryl; (37) —(CH₂)ᵣCONR^{B}R^{C}, where r is an integer of from zero to four and where each R^{B} and R^{C} is independently selected from the group consisting of (a) hydrogen, (b) C₁₋₆ alkyl, (c) C₄₋₁₈ aryl, and (d) C₁₋₆ alk-C₄₋₁₈ aryl; (38) —(CH₂)ᵣSO₂R^{D}, where r is an integer of from zero to four and where R^{D} is selected from the group consisting of (a) C₁₋₆ alkyl, (b) C₄₋₁₈ aryl, and (c) C₁₋₆ alk-C₄₋₁₈ aryl; (39) —(CH₂)ᵣSO₂NR^{E}R^{F}, where r is an integer of from zero to four and where each of R^{E} and R^{F} is, independently, selected from the group consisting of (a) hydrogen, (b) C₁₋₆ alkyl, (c) C₄₋₁₈ aryl, and (d) C₁₋₆ alk-C₄₋₁₈ aryl; (40) —(CH₂)ᵣNR^{G}R^{H}, where r is an integer of from zero to four and where each of R^{G} and R^{H} is, independently, selected from the group consisting of (a) hydrogen, (b) an N-protecting group, (c) C₁₋₆ alkyl, (d) C₂₋₆ alkenyl, (e) C₂₋₆ alkynyl, (f) C₄₋₁₈ aryl, (g) C₁₋₆ alk-C₄₋₁₈ is aryl, (h) C₃₋₈ cycloalkyl, and (i) C₁₋₆ alk-C₃₋₈ cycloalkyl, wherein in one embodiment no two groups are bound to the nitrogen atom through a carbonyl group or a sulfonyl group; (41) thiol; (42) perfluoroalkyl (e.g., an alkyl group, as defined herein, having each hydrogen atom substituted with a fluorine atom); (43) perfluoroalkoxy (e.g., —ORf, in which Rf is an alkyl group, as defined herein, having each hydrogen atom substituted with a fluorine atom); (44) aryloxy (e.g., —OAr, where Ar is an optionally substituted aryl group, as described herein); (45) cycloalkoxy (e.g., —OCy, in which Cy is a cycloalkyl group, as defined herein); (46) cycloalkylalkoxy (e.g., —OA^{L}Cy, in which A^{L} is an alkylene group and Cy is a cycloalkyl group, as defined herein); and (47) arylalkoxy (e.g., —OA^{L}Ar, in which A^{L} is an alkylene group and Ar is an aryl group, as defined herein). In particular embodiments, an unsubstituted aryl group is a C₄₋₁₈, C₄₋₁₄, C₄₋₁₂, C₄₋₁₀, C₆₋₁₈, C₆₋₁₄, C₆₋₁₂, or C₆₋₁₀ aryl group.

By “arylene” is meant a multivalent (e.g., bivalent, trivalent, tetravalent, etc.) form of an aryl group, as described herein. Exemplary arylene groups include phenylene, naphthylene, biphenylene, triphenylene, diphenyl ether, acenaphthenylene, anthrylene, or phenanthrylene. In some embodiments, the arylene group is a C₄₋₁₈, C₄₋₁₄, C₄₋₁₂, C₄₋₁₀, C₆₋₁₈, C₆₋₁₄, C₆₋₁₂, or C₆₋₁₀ arylene group. The arylene group can be branched or unbranched. The arylene group can also be substituted or unsubstituted. For example, the arylene group can be substituted with one or more substitution groups, as described herein for aryl.

By “aryloxy” is meant —OR, where R is an optionally substituted aryl group, as described herein. In some embodiments, an unsubstituted aryloxy group is a C₄₋₁₈ or C₆₋₁₈ aryloxy group.

By “aryloxycarbonyl” is meant an aryloxy group, as defined herein, that is attached to the parent molecular group through a carbonyl group. In some embodiments, an unsubstituted aryloxycarbonyl group is a C₅₋₁₉ aryloxycarbonyl group.

By “aryloyl” is meant an aryl group that is attached to the parent molecular group through a carbonyl group. In some embodiments, an unsubstituted aryloyl group is a C₇₋₁₁ aryloyl group.

By “azido” is meant an —N₃ group.

By “azo” is meant an —N═N— group.

By “carbonyl” is meant a —C(O)— group, which can also be represented as >C═O.

By “carboxyaldehyde” is meant a —C(O)H group.

By “carboxyl” is meant a —CO₂H group.

By “cyano” is meant a —CN group.

By “cycloalkyl” is meant a monovalent saturated or unsaturated non-aromatic cyclic hydrocarbon group of from three to eight carbons, unless otherwise specified, and is exemplified by cyclopropyl, cyclobutyl, cyclopentyl, cyclohexyl, cycloheptyl, bicyclo[2.2.1.]heptyl and the like. The cycloalkyl group can also be substituted or unsubstituted. For example, the cycloalkyl group can be substituted with one or more groups including those described herein for alkyl.

By “cycloalkoxy” is meant a cycloalkyl group, as defined herein, attached to the parent molecular group through an oxygen atom.

By “halo” is meant F, Cl, Br, or I.

By “haloalkyl” is meant an alkyl group, as defined herein, substituted with one or more halo.

By “heteroalkyl” is meant an alkyl group, as defined herein, containing one, two, three, or four non-carbon heteroatoms (e.g., independently selected from the group consisting of nitrogen, oxygen, phosphorous, sulfur, or halo).

By “heteroalkylene” is meant a divalent form of an alkylene group, as defined herein, containing one, two, three, or four non-carbon heteroatoms (e.g., independently selected from the group consisting of nitrogen, oxygen, phosphorous, sulfur, or halo).

By “heteroaryl” is meant a subset of heterocyclyl groups, as defined herein, which are aromatic, i.e., they contain 4n+2 pi electrons within the mono- or multicyclic ring system.

By “heterocyclyl” is meant a 5-, 6- or 7-membered ring, unless otherwise specified, containing one, two, three, or four non-carbon heteroatoms (e.g., independently selected from the group consisting of nitrogen, oxygen, phosphorous, sulfur, or halo). The 5-membered ring has zero to two double bonds and the 6- and 7-membered rings have zero to three double bonds. The term “heterocyclyl” also includes bicyclic, tricyclic and tetracyclic groups in which any of the above heterocyclic rings is fused to one, two, or three rings independently selected from the group consisting of an aryl ring, a cyclohexane ring, a cyclohexene ring, a cyclopentane ring, a cyclopentene ring, and another monocyclic heterocyclic ring, such as indolyl, quinolyl, isoquinolyl, tetrahydroquinolyl, benzofuryl, benzothienyl and the like. Heterocyclics include thiiranyl, thietanyl, tetrahydrothienyl, thianyl, thiepanyl, aziridinyl, azetidinyl, pyrrolidinyl, piperidinyl, azepanyl, pyrrolyl, pyrrolinyl, pyrazolyl, pyrazolinyl, pyrazolidinyl, imidazolyl, imidazolinyl, imidazolidinyl, pyridyl, homopiperidinyl, pyrazinyl, piperazinyl, pyrimidinyl, pyridazinyl, oxazolyl, oxazolidinyl, isoxazolyl, isoxazolidiniyl, morpholinyl, thiomorpholinyl, thiazolyl, thiazolidinyl, isothiazolyl, isothiazolidinyl, indolyl, quinolinyl, isoquinolinyl, benzimidazolyl, benzothiazolyl, benzoxazolyl, furyl, thienyl, thiazolidinyl, isothiazolyl, isoindazoyl, triazolyl, tetrazolyl, oxadiazolyl, uricyl, thiadiazolyl, pyrimidyl, tetrahydrofuranyl, dihydrofuranyl, tetrahydrothienyl, dihydrothienyl, dihydroindolyl, tetrahydroquinolyl, tetrahydroisoquinolyl, pyranyl, dihydropyranyl, dithiazolyl, benzofuranyl, benzothienyl, and the like.

By “hydroxyl” is meant —OH.

By “hydroxyalkyl” is meant an alkyl group, as defined herein, substituted by one to three hydroxyl groups, with the proviso that no more than one hydroxyl group may be attached to a single carbon atom of the alkyl group and is exemplified by hydroxymethyl, dihydroxypropyl, and the like.

By “nitro” is meant an —NO₂ group.

By “nitroalkyl” is meant an alkyl group, as defined herein, substituted by one to three nitro groups.

By “nitroso” is meant an —NO group.

By “oxo” is meant an ═O group.

By “oxy” is meant —O—.

By “perfluoroalkyl” is meant an alkyl group, as defined herein, having each hydrogen atom substituted with a fluorine atom. Exemplary perfluoroalkyl groups include trifluoromethyl, pentafluoroethyl, etc.

By “perfluoroalkylene” is meant an alkylene group, as defined herein, having each hydrogen atom substituted with a fluorine atom. Exemplary perfluoroalkylene groups include difluoromethylene, tetrafluoroethylene, etc.

By “perfluoroalkyleneoxy” is meant a perfluoroalkylene group, as defined herein, having an oxy group attached to either end of the perfluoroalkylene group. Exemplary perfluoroalkylene groups include, e.g., —OC_{f}F_{2f}— or —C_{f}F_{2f}O—, where f is an integer from about 1 to 5, and 2f is an integer that is 2 times f (e.g., difluoromethyleneoxy, tetrafluoroethyleneoxy, etc.).

By “perfluoroalkoxy” is meant an alkoxy group, as defined herein, having each hydrogen atom substituted with a fluorine atom.

By “phosphono” is meant a —P(O)(OH)₂ group.

By “phosphonoyl” is meant a —P(O)H— group.

By “phosphoric ester” is meant a —O—PO(OH)₂ group.

By “phosphoryl” is meant a —P(O)<group.

By “protecting group” is meant any group intended to protect a reactive group against undesirable synthetic reactions. Commonly used protecting groups are disclosed in “Greene's Protective Groups in Organic Synthesis,” John Wiley & Sons, New York, 2007 (4th ed., eds. P. G. M. Wuts and T. W. Greene), which is incorporated herein by reference. O-protecting groups include an optionally substituted alkyl group (e.g., forming an ether with reactive group O), such as methyl, methoxymethyl, methylthiomethyl, benzoyloxymethyl, t-butoxymethyl, etc.; an optionally substituted alkanoyl group (e.g., forming an ester with the reactive group O), such as formyl, acetyl, chloroacetyl, fluoroacetyl (e.g., perfluoroacetyl), methoxyacetyl, pivaloyl, t-butylacetyl, phenoxyacetyl, etc.; an optionally substituted aryloyl group (e.g., forming an ester with the reactive group O), such as —C(O)—Ar, including benzoyl; an optionally substituted alkylsulfonyl group (e.g., forming an alkylsulfonate with reactive group O), such as —SO₂—R^{S1}, where R^{S1} is optionally substituted C₁₋₁₂ alkyl, such as mesyl or benzylsulfonyl; an optionally substituted arylsulfonyl group (e.g., forming an arylsulfonate with reactive group O), such as —SO₂—R^{S4}, where R^{S4} is optionally substituted C₄₋₁₈ aryl, such as tosyl or phenylsulfonyl; an optionally substituted alkoxycarbonyl or aryloxycarbonyl group (e.g., forming a carbonate with reactive group O), such as —C(O)—ORT, where RT is optionally substituted C₁₋₁₂ alkyl or optionally substituted C₄₋₁₈ aryl, such as methoxycarbonyl, methoxymethylcarbonyl, t-butyloxycarbonyl (Boc), or benzyloxycarbonyl (Cbz); or an optionally substituted silyl group (e.g., forming a silyl ether with reactive group O), such as —Si—(R^{T2})₃, where each R^{T2} is, independently, optionally substituted C₁₋₁₂ alkyl or optionally substituted C₄₋₁₈ aryl, such as trimethylsilyl, t-butyldimethylsilyl, or t-butyldiphenylsilyl. N-protecting groups include, e.g., formyl, acetyl, benzoyl, pivaloyl, t-butylacetyl, alanyl, phenylsulfonyl, benzyl, Boc, and Cbz. Such protecting groups can employ any useful agent to cleave the protecting group, thereby restoring the reactivity of the unprotected reactive group.

By “salt” is meant an ionic form of a compound or structure (e.g., any formulas, compounds, or compositions described herein), which includes a cation or anion compound to form an electrically neutral compound or structure. Salts are well known in the art. For example, non-toxic salts are described in Berge S M et al., “Pharmaceutical salts,” J. Pharm. Sci. 1977 January; 66(1):1-19; and in “Handbook of Pharmaceutical Salts: Properties, Selection, and Use,” Wiley-VCH, April 2011 (2nd rev. ed., eds. P. H. Stahl and C. G. Wermuth. The salts can be prepared in situ during the final isolation and purification of the compounds of the invention or separately by reacting the free base group with a suitable organic acid (thereby producing an anionic salt) or by reacting the acid group with a suitable metal or organic salt (thereby producing a cationic salt). Representative anionic salts include acetate, adipate, alginate, ascorbate, aspartate, benzenesulfonate, benzoate, bicarbonate, bisulfate, bitartrate, borate, bromide, butyrate, camphorate, camphorsulfonate, chloride, citrate, cyclopentanepropionate, digluconate, dihydrochloride, diphosphate, dodecylsulfate, edetate, ethanesulfonate, fumarate, glucoheptonate, glucomate, glutamate, glycerophosphate, hemisulfate, heptonate, hexanoate, hydrobromide, hydrochloride, hydroiodide, hydroxyethanesulfonate, hydroxynaphthoate, iodide, lactate, lactobionate, laurate, lauryl sulfate, malate, maleate, malonate, mandelate, mesylate, methanesulfonate, methylbromide, methylnitrate, methylsulfate, mucate, 2-naphthalenesulfonate, nicotinate, nitrate, oleate, oxalate, palmitate, pamoate, pectinate, persulfate, 3-phenylpropionate, phosphate, picrate, pivalate, polygalacturonate, propionate, salicylate, stearate, subacetate, succinate, sulfate, tannate, tartrate, theophyllinate, thiocyanate, triethiodide, toluenesulfonate, undecanoate, valerate salts, and the like. Representative cationic salts include metal salts, such as alkali or alkaline earth salts, e.g., barium, calcium (e.g., calcium edetate), lithium, magnesium, potassium, sodium, and the like; other metal salts, such as aluminum, bismuth, iron, and zinc; as well as nontoxic ammonium, quaternary ammonium, and amine cations, including, but not limited to ammonium, tetramethylammonium, tetraethylammonium, methylamine, dimethylamine, trimethylamine, triethylamine, ethylamine, pyridinium, and the like. Other cationic salts include organic salts, such as chloroprocaine, choline, dibenzylethylenediamine, diethanolamine, ethylenediamine, methylglucamine, and procaine. Yet other salts include pharmaceutically acceptable salts, as described herein.

By “solvate” is meant a stabilized form of a compound or structure (e.g., any formulas, compounds, or compositions described herein, including anionic or cationic forms thereof) with one or more solvent molecules. Such forms can be stabilized by any useful interaction, such as electrostatic forces, van der Waals forces, or hydrogen bond formation. Exemplary solvates include hydrates (including one or more water molecules).

By “sulfinyl” is meant an —S(O)— group.

By “sulfo” is meant an —S(O)₂OH group.

By “sulfonyl” is meant an —S(O)₂— group.

By “anhydrate” is meant a form of a compound or structure (e.g., any formulas, compounds, or compositions described herein) generally lacking solvent molecules.

By “attaching,” “attachment,” or related word forms is meant any covalent or non-covalent bonding interaction between two components. Non-covalent bonding interactions include, without limitation, hydrogen bonding, ionic interactions, halogen bonding, electrostatic interactions, π bond interactions, hydrophobic interactions, inclusion complexes, clathration, van der Waals interactions, and combinations thereof.

By “pharmaceutically acceptable excipient” is meant any ingredient other than a compound or structure (e.g., any formulas, compounds, or compositions described herein) and having the properties of being nontoxic and non-inflammatory in a subject. Exemplary, non-limiting excipients include adjuvants, antiadherents, antioxidants, binders, carriers, coatings, compression aids, diluents, disintegrants, dispersing agents, dyes (colors), emollients, emulsifiers, fillers (diluents), film formers or coatings, flavors, fragrances, glidants (flow enhancers), isotonic carriers, lubricants, preservatives, printing inks, solvents, sorbents, stabilizers, suspensing or dispersing agents, surfactants, sweeteners, waters of hydration, or wetting agents. Any of the excipients can be selected from those approved, for example, by the United States Food and Drug Administration or other governmental agency as being acceptable for use in humans or domestic animals. Exemplary excipients include, but are not limited to alcohol, butylated hydroxytoluene (BHT), calcium carbonate, calcium phosphate (dibasic), calcium stearate, cellulose, croscarmellose, cross-linked polyvinyl pyrrolidone, citric acid, crospovidone, cysteine, ethylcellulose, gelatin, glucose, glycerol, hydroxypropyl cellulose, hydroxypropyl methylcellulose, lactated Ringer's solution, lactose, magnesium carbonate, magnesium stearate, maltitol, maltose, mannitol, methionine, methylcellulose, methyl paraben, microcrystalline cellulose, polyethylene glycol, polyol, polyvinyl pyrrolidone, povidone, pregelatinized starch, propyl paraben, retinyl palmitate, Ringer's solution, shellac, silicon dioxide, sodium carboxymethyl cellulose, sodium chloride injection, sodium citrate, sodium saccharine, sodium starch glycolate, sorbitol, starch (corn), stearic acid, stearic acid, sucrose, talc, talcum, titanium dioxide, vegetable oil, vitamin A, vitamin E, vitamin C, water, and xylitol.

By “pharmaceutically acceptable salt” is meant a salt that is, within the scope of sound medical judgment, suitable for use in contact with the tissues of humans and animals without undue toxicity, irritation, allergic response and the like and are commensurate with a reasonable benefit/risk ratio.

By “isomer” is meant a molecule having the same molecular formula as the reference molecule. Exemplary isomers include stereoisomers, diastereomers, enantiomers, geometric isomers, tautomers, as well as mixtures thereof.

As used herein, the terms “top,” “bottom,” “upper,” “lower,” “above,” and “below” are used to provide a relative relationship between structures. The use of these terms does not indicate or require that a particular structure must be located at a particular location in the apparatus.

Other features and advantages of the invention will be apparent from the following description and the claims.

### BRIEF DESCRIPTION OF THE DRAWINGS

FIG. 1A is a ball-and-stick depiction of a representative Eu hexanuclear cluster of an exemplary EuDOBDC MOF composition. FIG. 1B is a ball-and-stick depiction of the octahedral cage. Hydrogen atoms and pore solvent molecules have been omitted for clarity; atom color scheme: M=light grey (Eu); C=medium grey, O=dark grey.

FIG. 2A is a graph of N₂ sorption isotherms for compounds 1-4 measured at 77 K. FIG. 2B is a graph of powder X-ray diffraction (PXRD) data for pristine and NOₓ exposed compound 1 after 1 hour and after 24 hours.

FIG. 3 shows Fourier transform infrared (FT-IR) spectra for pristine compound 1 and after 24 hr exposure to NOₓ.

FIG. 4 shows investigated chemical configurations of NO interaction with the DOBDC ligand. Provided binding configurations include: (a) a DOBDC ligand, (b) a nitro group, (c) NO at a hydroxyl site, (d) NO at a carbonyl site, (e) NO₂ at a hydroxyl site, and (f) NO₂ at a carbonyl site.

FIG. 5A shows thermogravimetric-mass spectrometry (TGA-MS) analysis of compound 1. FIG. 5B shows TGA-MS analysis of compound 1 after NOₓ exposure for 24 hours.

FIG. 6 shows calculated binding energies of H₂O (circle) and NO₂ (diamond) adsorbed to unsaturated metal sites of Y, Eu, Tb, and Yb.

FIG. 7A shows an adsorption geometry for an individually adsorbed H₂O at an unsaturated Y metal site. FIG. 7B shows an adsorption geometry for an individually adsorbed NO₂ at an unsaturated Y metal site. Atom scheme: Y (large, light gray spheres), 0 (medium, black spheres), C (medium, dark gray spheres), H (small, white spheres), and N (medium black sphere with white dashed outline).

FIG. 8A shows photographs of the powders of compound 1 under ambient light (left) and UV light (right) either (top) before or (bottom) after NOₓ exposure for 24 hours. FIG. 8B shows PL emission spectra for compound 1 either before (labeled “pristine”) or after NOₓ exposure for 24 hours.

FIG. 9A shows photographs of the powders of compound 4 under ambient light (left) and UV light (right) either (top) before or (bottom) after NOₓ exposure for 24 hours. FIG. 9B shows PL emission spectra for compound 4 either before (labeled “pristine”) or after NOₓ exposure for 24 hours.

FIG. 10 shows calculated optical absorption spectra for activated YDOBDC (labeled “activated”), YDOBDC+H₂O (labeled “H₂O”), and YDOBDC+NO₂ (labeled “NO₂”).

FIG. 11A shows partial charge densities (gray isosurface) of KS orbitals for transitions B, between orbitals 560-569 with the participation of adsorbed H₂O represented by a dashed gray circle. FIG. 11B shows partial charge densities (gray isosurface) of KS orbitals for transitions B, between orbitals 559-572 with participation of adsorbed NO₂ represented by a dashed gray circle.

### DETAILED DESCRIPTION OF THE INVENTION

Sensing and detection of acid gases (e.g., NOₓ and/or SOₓ) is very relevant for industrial and environmental purposes, since the gases are notorious polluters contributing to the formation of smog and acid rain. The present invention is directed to a method of detecting an acid gas based upon the photoluminescence response to acid gases of a metal-organic framework (MOF) materials platform based on rare earth metal ions (e.g., RE=Eu, Nd, Yb, Y, Tb) and a linear dicarboxylic acid (e.g., 2,5-dihydroxyterephtalic acid) and related MOF materials. For example, the composition of this materials family can be easily extended to the entire lanthanide metal series (e.g., La, Ce, Pr, Pm, Sm, Gd, Dy, Ho, Er, Tm, Lu) and other transition metals. Also, the composition can include one metal or a combination of two or more different metals. Other types of coordinating ligands can also be used. From a commercial standpoint, this technology could be pertinent to the automotive industry, as directly related to stringent exhaust gas regulations. In particular, many manufacturing and processing industries (e.g., petrochemical and automotive industries) need acid gas sensors and are actively pursuing such sensors for these applications. Interests include determining the content of acid gases in streams for refinery processing; and developing onboard sensors of acid gases and interference with engine performance for the automotive industry. The ability of adsorbed NOₓ in this class of materials to nearly extinguish the emission from each of these MOFs enables their use in optical gas sensors.

### MOF Compositions

The MOF compositions herein can include any useful metal (e.g., a metal ion). The composition can include one metal or a combination of two or more different metals. In addition, the composition can include the same metal having different coordination geometries. Exemplary metals include a rare earth metal, e.g., cerium (Ce), dysprosium (Dy), erbium (Er), europium (Eu), gadolinium (Gd), holmium (Ho), lanthanum (La), lutetium (Lu), neodymium (Nd), praseodymium (Pr), promethium (Pm), samarium (Sm), scandium (Sc), terbium (Tb), thulium (Tm), ytterbium (Yb), and yttrium (Y); or a transition metal, e.g., zinc (Zn), zirconium (Zr), titanium (Ti), hafnium (Hf), iridium (Ir), or copper (Cu).

Furthermore, the composition can include one or more metal clusters. Each cluster, in turn, can include a metal ion with one or more ligands. Within a cluster, if a plurality of metal ions is present within the same cluster, each metal ion can be the same or different. Between clusters, the metal ion of a first cluster can be the same or different than the metal ion of a second cluster. Each metal cluster can be the same or different. Exemplary differences can be a different element, a different coordination geometry, a different combination of ligand bridging or chelating, a different ligand, etc.

In one non-limiting embodiment, the cluster includes a plurality of metal ions, in which each metal ion is coordinated to one or more ligands (e.g., a bridging ligand, a chelating ligand, a bridging/chelating ligand). Exemplary ligands include hydroxyl (e.g., μₙ—OH, in which n is 1, 2, 3, etc.), a monodentate ligand, a bidentate ligand (e.g., a bidentate bridging ligand, a bis-bidentate bridging ligand, a bidentate chelating ligand, or a bis-bidentate chelating ligand), a tridentate ligand (e.g., a tridentate bridging ligand or a tridentate chelating ligand), a tetradentate ligand (e.g., a tetradentate bridging ligand or a tetradentate chelating ligand), etc.

In some embodiments, the cluster coordinates with both a monodentate ligand and a bidentate ligand. In other embodiments, the cluster coordinates with a plurality of monodentate ligands and a plurality of bidentate ligands. The clusters and ligands can form any useful network (e.g., a periodic network, in one instance characterized by a tetragonal crystal structure).

Ligands can have any useful structure. In one non-limiting embodiment, the ligand has the structure of (L¹)ₘ-R^{L}-(L²)ₙ, where each of L¹ and L² is, independently, a reactive group; where R^{L} is a linker; and where each of m and n is, independently, 1, 2, 3, 4, 5, 6, or one of m or n is 0. For instance, if m and n are both one, then the ligand is a bivalent ligand (e.g., L¹-R^{L}-L²). In another instance, if m is 1 and n is 2, then the ligand is a trivalent ligand (e.g., L¹-R^{L}-(L²)₂ or L¹-R^{L}<L^{2a}L^{2b}, in which each L² is the same or different or in which L^{2a} and L^{2b} are the same or different).

L¹ and L² can be any useful reactive group, such as any useful for forming a metal bond (e.g., a coordinate bond, a covalent bond, etc.). Exemplary reactive groups can include carboxyl, heterocyclyl, amino, phosphoryl, sulfonyl, as well as anionic forms thereof (e.g., carboxylate, azolate (e.g., such as imidazolate, pyrazolate, triazolate, tetrazolate), phosphate, sulfonate, sulfate, etc.), salts thereof, or esters thereof.

The ligand can have any useful linker (e.g., R^{L}). Exemplary linkers can include an optionally substituted aryl (e.g., optionally substituted arylene), optionally substituted heteroaryl (e.g., optionally substituted heteroarylene), an optionally substituted alkyl (e.g., optionally substituted alkylene), or an optionally substituted heteroalkyl (e.g., optionally substituted heteroalkylene). Optional substitutions can include one or more of the following on a backbone (e.g., an arylene or alkylene backbone): hydroxyl, optionally substituted alkyl, haloalkyl, hydroxyalkyl, optionally substituted alkoxy (e.g., methoxy, ethoxy, benzyloxy, etc.), optionally substituted cycloalkyl, optionally substituted cycloalkoxy, optionally substituted aryl, optionally substituted aryloxy, halo, carboxyl, azido, cyano, nitro, amino, aminoalkyl, or carboxyaldehyde, as well as any optional substituents described herein for alkyl and aryl.

Exemplary linkers can include an optionally substituted phenylene, optionally substituted dithieno[3,2-b; 2′,3′-d]-thiophene, optionally substituted 2,2′-bipyridyl, optionally substituted terphenylene (in ortho, meta, or para forms), and an optionally substituted biphenylene.

The MOF can include any useful metal (e.g., metal atom, metal ion, or metal cluster) in combination with any useful ligand (e.g., any described herein). Further non-limiting, exemplary ligands include 3,3′,5,5′-azobenzenetetracarboxylate (ADB⁴⁻); 5,5′-(9,10-anthracenediyl)di-isophthalate (ADIP⁴⁻); adamantane-1,3,5,7-tetracarboxylate (ATC⁴⁻); 4,4′,4″-(benzene-1,3,5-triyl-tris(benzene-4,1-diyl))tribenzoate (BBC³⁻ or TCBB³⁻); 1,4-benzenedicarboxylate (BDC²⁻); BDC-(X)²— or BDC-(X)₂², where each X is, independently, alkyl, halo, hydroxyl, nitro, amino, carboxyl, alkoxy, cycloalkoxy, aryloxy, benzyloxy (e.g., 2-amino-1,4-benzenedicarboxylate (BDC-NH₂²⁻) or 2,5-diamino-1,4-benzenedicarboxylate (BDC-(NH₂)₂²⁻); 5,5′,5″-((((benzene-1,3,5-triyltris(benzene-4,1-diyl))tris(ethyne-2,1-diyl))-tris(benzene-4,1-diyl))tris(ethyne-2,1-diyl))triisophthalate] (BHEHPI⁶⁻); 5,5′,5″-(benzene-1,3,5-triyl-tris(buta-1,3-diyne-4,1-diyl)) triisophthalate (BHEI⁶⁻); 5,5′,5″-(((benzene-1,3,5-triyl-tris(ethyne-2,1-diyl))tris(benzene-4,1-diyl))tris(buta-1,3-diyne-4,1-diyl))triisophthalate (BNETPI⁶⁻); 4,4′-biphenyl dicarboxylate (BPDC²⁻); 2,2′-bipyridine-5,5′-dicarboxylate (BPYDC²⁻); 4,4′,4″-benzene-1,3,5-triyl-tribenzoate (BTB³⁻); 1,3,5-benzenetricarboxylate or 1,2,4-benzenetricarboxylate (BTC³⁻); 4,4′,4″-(benzene-1,3,5-triyl-tris(ethyne-2,1-diyl))tribenzoate (BTE³⁻); 5,5′,5″-(benzene-1,3,5-triyl-tris(ethyne-2,1-diyl))triisophthalate (BTEI⁶⁻); 5′,5″″,5′″″″-(benzene-1,3,5-triyl-tris(ethyne-2,1-diyl))tris (([1,1′:3′,1″-terphenyl]-4,4″-dicarboxylate)) (BTETCA³⁻); 4,4′,4″-(benzene-1,3,5-triyl)tris (pyrazol-1-ide) (BTP³⁻); 5,5′,5″-(benzene-1,3,5-triyl-tris(benzene-4,1-diyl))triisophthalate (BTPI⁶⁻); 5,5′,5″-(benzene-1,3,5-triyl-tris(biphenyl-4,4′-diyl))triisophthalate (BTTI⁶⁻); 3,3′-difluoro-biphenyl-4,4′-dicarboxylate (DFBPDC²⁻); 2,5-dioxido-1,4-benzenedicarboxylate (DOBDC⁴⁻); 4,6-dioxido-1,3-benzenedicarboxylate (m-DOBDC⁴⁻); 4,4′-dioxidobiphenyl-3,3′-dicarboxylate (DOBPDC⁴⁻); dioxidoterephthalate (DOT²⁻); 4,4′-([2,2′-bipyridine]-5,5′-diyl) dibenzoate (DPBPyDC²⁻); 3-fluoro-biphenyl-4,4′-dicarboxylate (FBPDC²⁻); 2-fluoro-4-(1H-tetrazol-5-yl)benzoate (FTZB²⁻); 3-fluoro-4′-(1H-tetrazol-5-yl)biphenyl-4-carboxylate (FTZBP²⁻); imidazoledicarboxylate (HImDC³⁻); 1,4-naphthalenedicarboxylate (NDC²⁻); 5,5′,5″-((benzene-1,3,5-triyl-tris(benzene-4,1-diyl))tris(ethyne-2,1-diyl))triisophthalate (PTEI⁶⁻); 3,5-pyridinedicarboxylate or 2,5-pyridinedicarboxylate (PyDC²⁻); 4,4′,4″-(1,3,5-triazine-2,4,6-triyl)tribenzoate (TATB³⁻); 1,3,6,8-tetrakis(p-benzoate)pyrene (TBAPy⁴⁻); 2,4,6-trihydroxy-1,3,5-benzenetrisulfonate (THBTS³⁻); tris(4-(1H-imidazol-1-yl)phenyl)amine (TIPA³⁻); 5,5′,5″-((benzene-1,3,5-tricarbonyl) tris(azanediyl))triisophthalate (TPBTM⁶⁻); 5,5′,5″-(((benzene-1,3,5-triyl-tris(ethyne-2,1-diyl)) tris(benzene-4,1-diyl))tris(ethyne-2,1-diyl))triisophthalate (TTEI⁶⁻); and 4-(1H-tetrazol-5-yl)benzoate (TZB²⁻); each of which may optionally include one or more counterions (e.g., one or more counteranions or countercations), as well as a cation thereof, an anion thereof, a protonated form thereof, a salt thereof, or an ester thereof.

Exemplary reagents to install a ligand include, e.g., oxalic acid; fumaric acid; adamantanetetracarboxylic acid (H₄ATC); adamantanetetrabenzoic acid (H₄ATB); 9,10-anthracenedicarboxylic acid (H₄ADB); acetylene dicarboxylic acid (H₂ADC); 1,3,5-tris(4′-carboxy[1,1′-biphenyl]-4-yl)benzene (H₃BBC); terephthalic acid and optionally substituted forms thereof (e.g., H₂BDC or H₂BDC-(X) or H₂BDC-(X)₂, in which X can be optionally substituted alkyl, halo, hydroxyl, nitro, amino, carboxyl, optionally substituted alkoxy, optionally substituted cycloalkoxy, or optionally substituted aryloxy); biphenyl-3,4′,5-tricarboxylic acid (H₃BHTC); biphenyl-3,3′,5,5′-tetracarboxylic acid (H₄BPTC); 1,3,5-tris(4-carboxy phenyl) benzene (H₃BTB); trimesic acid (H₃BTC); 1,3,5-triscarboxyphenyl ethynylbenzene (H₃BTE); 2,5-dihydroxyterephthalic acid (H₄DOBDC); 2,5-dihydroxy-1,4-benzenedicarboxylic acid (H₄DOT); glycine-alanine (Gly-Ala); imidazole (Im); methylimidazole (mIm); 3,3′,5,5′-tetracarboxydiphenylmethane (H₄MDIP); 2-methylimidazole (HMIM); methane tetrabenzoic acid (H₄MTB); 2,6-naphthalenedicarboxylic acid (2,6-H₂NDC); 5′-(4-carboxyphenyl)-[1,1′:3′,1″-terphenyl]-3,3″,5,5″-tetracarboxylic acid (H₅PTPCA); 4,4′,4″-s-triazine-2,4,6-triyl-tribenzoic acid (H₃TATB); 1,2,4,5-tetrakis(4-carboxyphenyl)benzene (H₄TCPB); [1,1′:4′,1″ ]terphenyl-3,3′,5,5′-tetracarboxylic acid (H₄TPTC), as well as optionally substituted forms of any of these (e.g., optional substitutions as provided for alkyl or aryl herein).

Exemplary MOF compositions include EuDOBDC (Eu₆(μ₃—OH)(C₈H₄O₆)₅(C₈H₆O₆)₁ (H₂O)₆.24 H₂O or Eu₁₂(OH)₁₆(C₈H₅O₆)₄(C₈H₄O₆)₈); YDOBDC (Y₁₂(OH)₁₆(C₈H₅O₆)₄(C₈H₄O₆)₈); NdDOBDC (Nd₁₂(OH)₁₆(C₈H₅O₆)₄(C₈H₄O₆)₈); YbDOBDC (Yb₁₂(OH)₁₆(C₈H₅O₆)₄(C₈H₄O₆)₈); TbDOBDC (Tb₁₂(OH)₁₆(C₈H₅O₆)₄(C₈H₄O₆)₈); Nd_{0.67}Yb_{0.33}DOBDC ((Nd_{0.67}Yb_{0.33})₂(OH)₁₆ (C₈H₅O₆)₄(C₈H₄O₆)₈); Nd_{0.46}Yb_{0.54}DOBDC ((Nd_{0.46}Yb_{0.54})₁₂(OH)₁(C₈H₅O₆)₄(C₈H₄O₆)₈); UiO-66-DOBDC (Zr₆(μ₃—O)₄(μ₃—OH)₄(C₈H₄O₆)₆); UiO-66 (Zr₆(μ₃—O)₄(μ₃—OH)₄(BDC)₆ or Zr₆(μ₃—O)₄(μ₃—OH)₄(C₈H₄O₄)₆); UiO-67 (Zr₆(μ₃—O)₄(μ₃—OH)₄(BPDC)₆); NU-1000 (Zr₆(μ₃—O)₄(μ₃—OH)₄(OH)₄(H₂O)₄(TBAPy⁴⁻)₂); MOF-808 (Zr₆(μ₃—O)₄(μ₃—OH)₄(OH)₆(H₂O)₆(BTC)₂); and PCN-777 (Zr₆(μ₃—O)₄(μ₃—OH)₄(OH)₆(H₂O)₆(CO₂)₆ for benzene-1,4-dicarboxylate (BDC²⁻), 1,3,6,8-tetrakis(p-benzoate)pyrene (TBAPy⁴⁻), benzene-1,3,5-tricarboxylate (BTC³⁻), and biphenyldicarboxylate (BPDC²⁻).

In some embodiments of the MOF composition, the metal cluster exhibits a unique ligand binding mode. Specifically, 10 out of the 12 dicarboxylate bridging linkers of the exemplary RE-DOBDC MOFs described herein can bind in an anticipated bis-bidentate way, while the remaining 2 ligands coordinate to the metal ions in a monodentate fashion. This distinct behavior is likely correlated with the presence of the hydroxyl groups in the close proximity of the carboxylates. The presence of the bridging disorder can occur in the a-b plane of the structure and serve as a true disorder of the DOBDC ligand, in which the ligand bonds in a bidentate fashion on one cluster and bridges to the adjacent cluster to bond in a monodentate coordination. Each ligand residing in the a-b plane can bond in this manner with one side of the ligand as bidentate and the other side connecting as monodentate. This could allow for two possible positions of the ligand between clusters, the same ligand lying side-by-side one another in the a-b plane, and only one of the two possible orientations may ever be occupied at any given time. This slight alteration of the binding mode can propagate a shift in the alignment of the clusters. As a result, there may be a change in the symmetry from anticipated cubic crystal system to tetragonal.

In other embodiments, the remaining coordination sites may be occupied by a total of six water molecules per cluster. These water molecules can be removed by applying heat, in vacuum (120° C.), generating coordinatively unsaturated metal centers. Importantly, only a very limited set of MOFs are known to exhibit this desirable property, which is highly useful for a variety of applications that are pertinent to tuning guest-framework interactions.

The MOF composition can have any useful form. In one non-limiting instance, the MOF is provided as a particle having a diameter greater than about 10 nm (e.g., greater than about 20 nm, 30 nm, 40 nm, 50 nm, 60 nm, 70 nm, 80 nm, 90 nm, 100 nm, 125 nm, 150 nm, 200 nm, 300 nm, 500 nm, 750 nm, 1 m, 2 m, 5 m, 10 m, 20 m, or more) or of from about 10 nm to about 100 nm (e.g., from 10 nm to 50 nm, 20 nm to 50 nm, 20 nm to 100 nm, 30 nm to 100 nm, etc.). In another non-limiting instance, the MOF is provided as a crystal having a dimension of from about 1 m to about 60 m. The composition can be provided as packed particles, a gel (e.g., including a plurality of particles), a crystal, a dehydrated form, etc. The MOF composition can also be provided as any useful article, such as an adsorbent, a textile, an aerosol, a decontamination formulation, etc.

### Acid Gases

According to the present invention, the MOF can be employed to detect one or more acid gases. Exemplary acid gases include nitrogen oxide (e.g., NOₓ, such as NO or NO₂), sulfur oxide (e.g., SOₓ, such as SO₂), hydrogen sulfide (H₂S), carbon dioxide (CO₂), as well as mixtures thereof. Such acid gases can also include water in vapor form (e.g., steam). Such acid gases can be present in any sample, such as an air sample, a gas sample, a flue sample, an exhaust sample, a waste sample, etc.

NOₓ Adsorption and Optical Detection in Rare Earth Metal-Organic Frameworks

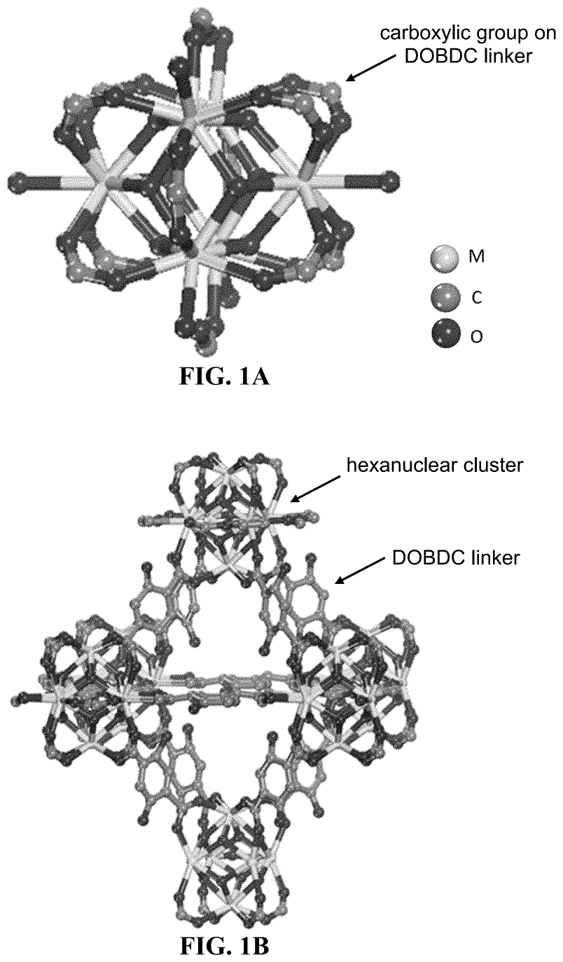

As an example of the invention, the multifunctionality of the isostructural rare earth (RE) based MOF family RE-DOBDC (RE=Y, Yb, Tb, Eu; DOBDC=2,5-dihydroxyterephthalic acid) as both adsorbents and gas sensing platforms was examined, as described below. See D. F. Sava Gallis et al., ACS Appl. Mater. Interfaces 9, 22268 (2017). The structure of an exemplary MOF composition, EuDOBDC, has been characterized and formulated by single crystal X-ray crystallography studies as having a Eu₂(OH)₁₆(C₈H₅O₆)₄(C₈H₄O₆)₈ unit cell with a tetragonal crystal structure. The structure is based on the pre-designed hydroxo-bridged cluster in which six metal atoms are coordinated by twelve DOBDC organic linkers, resulting in an overall 12-connected node, as shown in FIG. 1A. In this crystal structure, the Eu metal ions adopt both 8- and 9-coordination geometries. The material is also defined by a three periodic framework with octahedral cages of ˜10.4 Å diameter, as shown in FIG. 1B. Two binding coordinations are exhibited in a unit cell, (C₈H₄O₆)₈ and (C₈H₅O₆)₄. The (C₈H₄O₆) linker is coordinated in a bidentate fashion, having four carboxylic group O bound to an Eu atom. The linker coordination of (C₈H₅O₆) is bidentate for one carboxylic group but monodentate at the opposite end of the DOBDC. See D. F. Sava Gallis et al., ACS Appl. Mater. Interfaces 9, 22268 (2017) and D. J. Vogel et al., Phys. Chem. Chem. Phys. 21, 23085 (2019).

From a structural perspective, these materials are closely related to the UiO-66 family, as they are built from a hexanuclear metal cluster, akin to the one commonly encountered in Zr-based MOFs. See J. H. Cavka et al., J. Am. Chem. Soc. 130, 13850 (2008). Stability to the corrosive environment can be expected, as inferred by the previously determined robustness of related metal clusters. See A. M. Ebrahim et al., Langmuir 29, 168 (2013); A. M. Ebrahim et al., Microporous Mesoporous Mater. 188, 149 (2014); and G. W. Peterson et al., Angew. Chem. Int. Ed. 55, 6235 (2016). Unique to this system is the coordination geometry of the RE metal ions (8- and 9- vs 6-coordinate in UiO-66) that can lead to monodentate binding for one of the bridging DOBDC linkers. The removal of coordinated water molecules infer potential exploitation of coordinatively unsaturated metal sites.

Given the unique attributes of this isostructural material platform, multifunctionality as both adsorbents and gas sensing platforms is possible with the RE-DOBDC MOFs of the present invention. Therefore, a study was undertaken to (i) investigate NOₓ stability and/or preferential adsorption as a function of metal identity, and (ii) probe the effect on the photoluminescent properties unique to these MOFs as a function of guest loading. A fundamental understanding of the structure-property relationship of NOₓ adsorption in the RE-DOBDC materials platform was obtained via a combined experimental-molecular modeling study. The structural and thermal stability to humid NOₓ adsorption was characterized by a variety of experimental probes, including powder X-ray diffraction (PXRD), Fourier transform infrared spectroscopy (FT-IR), and thermogravimetric analyses coupled with mass spectrometry (TGA-MS). In particular, no structural change was noted following humid NOₓ exposure. Additionally, the photoluminescence properties on pre- and post-NOₓ exposure were monitored.

Complementary ab initio density functional theory (DFT) and classical DFT modeling studies were implemented to provide additional molecular level insights into the binding mechanism of NO₂ in these materials, as well as describe their impact on the photophysical properties. DFT simulations indicated that H₂O has a stronger affinity to bind with the metal center than NO₂, while NO₂ preferentially binds with the DOBDC ligands. Further modeling results indicate no change in binding energy across the RE elements investigated. Also, stabilization of the NO₂ and H₂O molecules following adsorption was noted, predicted to be due to hydrogen bonding between the framework ligands and the molecules and nanoconfinement within the MOF structure. This interaction also caused distinct changes in emission spectra, identified experimentally. Calculations indicated that this is due to the adsorption of NO₂ molecules onto the DOBDC ligand altering the electronic transitions and the resulting photoluminescent properties of the MOF, a feature that has potential applications in future sensing technologies.

### Synthesis of Exemplary RE-DOBDC MOFs

Methods of synthesizing exemplary RE-DOBDC MOF compositions are described below, as well as in U.S. patent application Ser. No. 15/994,904, filed May 31, 2018, and Ser. No. 16/201,224, filed Nov. 27, 2018; D. F. Sava Gallis et al., ACS Appl. Mater. Interfaces 9, 22268 (2017); and D. F. Sava Gallis et al., ACS Appl. Mater. Interfaces 11, 43270 (2019), each of which is incorporated herein by reference in its entirety. All reactant materials were purchased from commercially available sources and used without further purification.

Synthesis of YDOBDC (compound 1): A reaction mixture containing Y(NO₃)₃.6H₂O (0.1080 g, 0.311 mmol), 2,5-dihydroxyterephthalic acid (DOBDC, 0.0816 g, 0.412 mmol), 2-fluorobenzoic acid (2-FBA, 0.8640 g, 6.17 mmol), N,N′-dimethylformamide (DMF, 8 mL), H₂O (2 mL), and HNO₃ (0.6 mL, 3.5 M in DMF) was placed in a 20 mL scintillation vial and was heated to 115° C. for 60 h, at a rate of 1.5° C./min and cooled to room temperature at a cooling rate of 1° C./min; crystalline material resulted with ˜35% yield.

Synthesis of YbDOBDC (compound 2): A reaction mixture containing Yb(NO₃)₃.5H₂O (0.0780 g, 0.174 mmol), DOBDC (0.0544 g, 0.275 mmol), 2-FBA (0.1948 g, 1.39 mmol), DMF (8.8 mL), H₂O (2 mL), and HNO₃ (0.4 mL, 3.5 M in DMF) were placed in a 20 mL scintillation vial and heated to 115° C. for 60 h, at a rate of 1.5° C./min and cooled to room temperature at a cooling rate of 1° C./min; crystalline material resulted with ˜50% yield.

Synthesis of TbDOBDC (compound 3): A reaction mixture containing Tb(NO₃)₃.5H₂O (0.1224 g, 0.281 mmol), DOBDC (0.0816 g, 0.412 mmol), 2-FBA (0.8640 g, 6.17 mmol), DMF (8 mL), H₂O (2 mL), and HNO₃(0.6 mL, 3.5 M in DMF) were placed in a 20 mL scintillation vial, heated to 115° C. for 60 h, at a rate of 1.5° C./min, and cooled to room temperature at a cooling rate of 1° C./min; crystalline material resulted with ˜45% yield.

Synthesis of EuDOBDC (compound 4): A reaction mixture containing EuCl₃.6H₂O (0.0689 g, 0.087 mmol), DOBDC (0.0544 g, 0.087 mmol), 2-FBA (0.5760 g, 4.12 mmol), DMF (8 mL), H₂O (2 mL), and HNO₃ (0.6 mL, 3.5 M in DMF) were placed in a 20 mL scintillation vial, heated to 115° C. for 60 h, at a rate of 1.5° C./min, and cooled to room temperature at a cooling rate of 1° C./min; crystalline material resulted with ˜50% yield.

### Structural Characterization

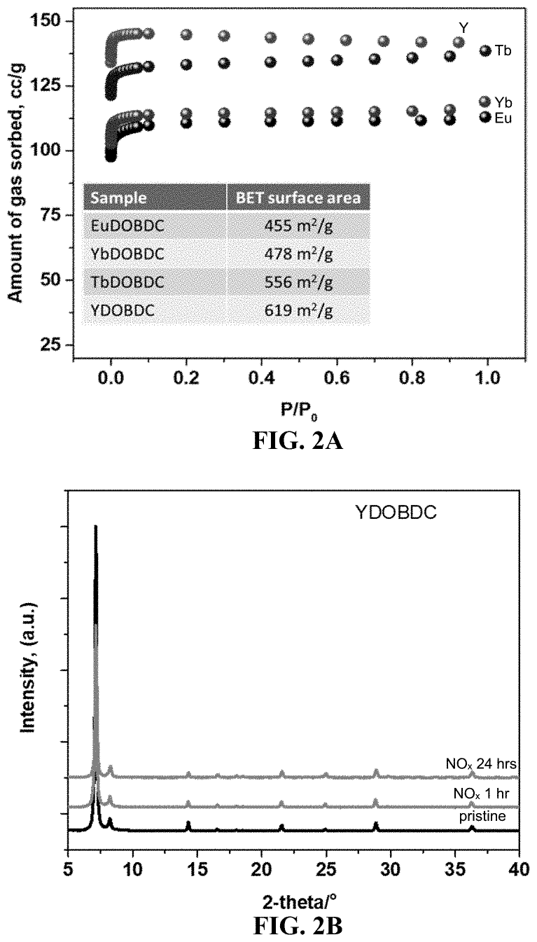

N₂ adsorption isotherms were measured at 77 K on compounds 1-4. As shown in FIG. 2A, all materials possess permanent porosity, displaying type I isotherms, typically observed in microporous materials. The calculated BET specific surface areas were on par with those found in a previous report. See D. F. Sava Gallis et al., ACS Appl. Mater. Interfaces 9, 22268 (2017).

The MOF materials were exposed to an 60% RH, ˜50 ppm NOₓ stream in an adsorption chamber at room temperature for 1 hr or 24 hr. All materials fully retained their crystallinity upon humid NOₓ exposure, as evidenced by PXRD patterns, as shown in FIG. 2B for compound 1 (YDOBDC). Interestingly, no definite changes in the peak signatures were noted upon guest loading. Due to largely related features upon NOₓ exposure across the series, compound 1 was chosen as a representative material. Its properties upon NOₓ adsorption are analyzed in detail below, with additional data provided for materials characterization for compounds 2, 3, and 4 as needed.

### Fourier Transform Infrared Spectroscopy and Thermogravimetric-Mass Spectrometry Analyses

FT-IR spectroscopy was employed to confirm the presence of the NOₓ-based species within the RE-DOBDC MOFs. This technique is highly sensitive, and it can distinguish among unique modes in the N—O bonding and potentially assess speciation. To be noted, it has been previously documented that NOₓ species are reactive with surfaces leading to nitrate and nitrite species, seen primarily with metal oxides systems. See J. A. Rodriguez et al., J. Phys. Chem. B 104, 319 (2000); and J. A. Rodriguez et al., J. Chem. Phys. 112, 9929 (2000).

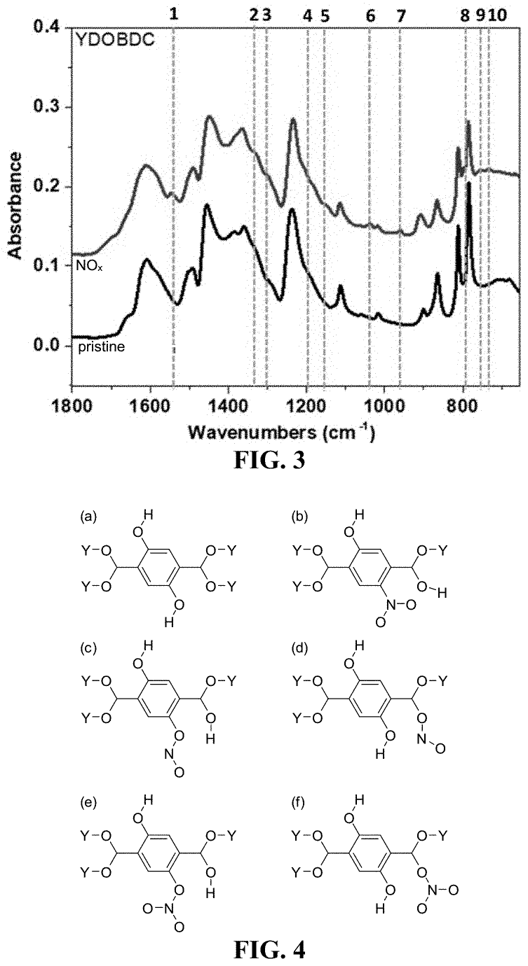

After 24 h of NOₓ exposure, several new bands at 1544, 1325, 1038, 960, 797, 755, and 733 cm⁻¹, corresponding to peaks 1, 2, 6, 7, 8, and 9 in FIG. 3A, began emerging in the FTIR spectra for all compounds. Assignments of these peaks for compound 1 are consistent with R—NO₂, organic nitrite R—ONO and organic nitrate R—ONO₂ species. Specifically, the peak at 1544 cm-1 is assigned to the asymmetric NO₂ stretch of the nitro groups, while that at 1325 cm-1 is associated with the symmetric NO₂ stretch; the peak at 960 cm-1 is associated with the aromatic C—N stretch of the nitro group, while that at 1038 cm-1 is assigned to the symmetric NO₂ stretch (R—ONO₂).

Additional new features, less pronounced, are observed and are mainly associated with organic nitrate, R—ONO₂, and organic nitrite, R—ONO, species. Unambiguous assignment is difficult due to overlapping features. Accordingly, the peak at 1296 cm-1 is attributed to the asymmetric NO₂ stretch in R—ONO₂, while those at 1206 and 1177 cm-1 correlate to the R—O stretches of organic nitrates/nitrite. Lastly, the peak at 797 cm-1 is assigned to the N—O stretch in R—ONO; that at 755 cm-1 to the NO₂ deformation in R—ONO₂/N—O stretch in R—ONO, and the feature at 733 cm-1 to the NO₂ deformation in R—ONO₂. Broadening of existing framework peaks is noted at 1609, 1449, and 909 cm⁻¹, indicative of guest-framework and change in linker environment upon NOₓ binding. Simulated NOₓ-linker interactions (binding configurations shown in FIG. 4) show the origin of the resonances observed in the experimental FT-IR spectra. All of the calculated configurations have favorable binding energies, indicating chemical feasibility of NOₓ binding to the ligand (for each of the interactions considered, the corresponding binding energies are provided in Table 1).

TABLE 1
Interaction distances and binding energies calculated for NO and
NO₂ interaction at hydroxyl and carbonyl sites on a DOBDC linker

| NOₓ/DOBDC | | | |
|---|---|---|---|
| binding | | Interaction | Binding energy |
| configuration | Gas | distance [Å] | [kJ/mol] |
| a | — | — | — |
| b | NO₂ | 1.44 | −106.48 |
| c | NO | 1.85 | −82.41 |
| d | NO | 1.72 | −82.24 |
| e | NO₂ | 1.94 | −41.03 |
| f | NO₂ | 1.61 | −104.60 |

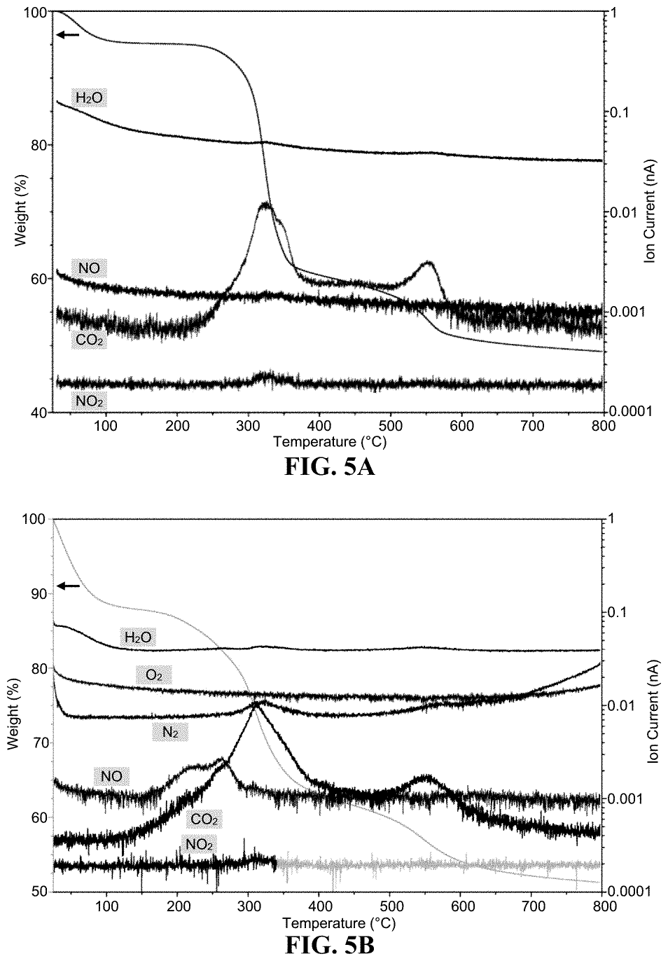

To gather additional insights into the NOₓ adsorption into the RE-DOBDC MOFs, TGA-MS was conducted on pristine and NOₓ-loaded materials. As a general note, all pristine analogs display thermal stability up to ˜275-300° C., when gradual framework degradation is observed, as evidenced by release of CO₂ from the linker, indicative of framework decomposition (as shown in FIG. 5A for compound 1). The guest-loaded materials display related thermal degradation profiles, as compared to the pristine materials (as shown in FIG. 5B for compound 1). Importantly, no NOₓ species are being desorbed in the 30-150° C. range, indicative of preferential/favored adsorption sites inside the MOF pore (rather than surface adsorption). NO gas is gradually released in the 150-300° C. range. This is consistent with the thermal decomposition of NO₂ and serves as additional supportive evidence to the IR studies to confirm the presence of NO₂ as the main component in the NOₓ speciation. See W. A. Rosser Jr. et al., J. Chem. Phys. 24, 493 (1956). This thermal event was consistently noted across all RE-DOBDC analogs. The overlapping NOₓ and CO₂ off gas events indicate that the framework may probably not withstand thermal recycling.

Computational Binding Energies for NOₓ and H₂O with Exemplary MOF Compositions

Density functional theory (DFT) studies were implemented to validate the experimental findings and provide additional insights on the molecular level details of NO₂ and H₂O adsorption in the RE-DOBDC material family. Individual H₂O and NO₂ gas molecules were placed at unsaturated metal sites to investigate the binding energies as a function of adsorbate and metal center.

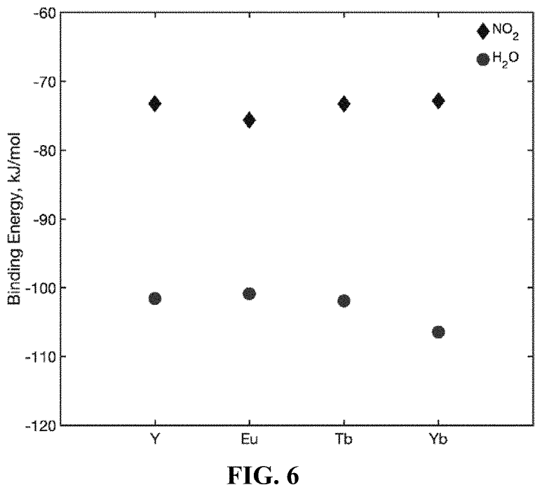

In general, NO₂ and H₂O bind with the current RE elements with strengths of −73.7±1.3 and −102.7±2.6 kJ/mol, respectively. Note, a negative binding energy indicates a decrease in system energy and therefore a more favorable binding configuration. The binding energy of H₂O and NO₂ for each RE analog of this MOF family is consistent across the RE elements studied, as shown in FIG. 6. This provides qualitative identification that H₂O is preferentially adsorbed at the RE metal sites within the RE-DOBDC MOF materials, as compared to the NO₂ molecule.

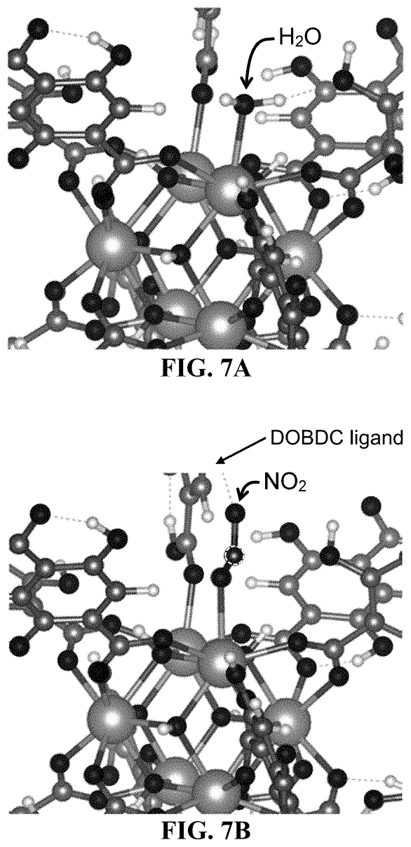

Differences in binding energy arise not just from the interaction of the metal with the guest molecule but also from secondary interactions, including H-bonding with nearby linkers. Structural details of the binding geometry can provide insight into these secondary factors. The lowest total energy calculated for gas orientations of H₂O and NO₂, respectively, at an unsaturated metal site are shown in FIGS. 7A-7B. For the strongest gas binding orientation, the unsaturated metal-gas (RE-O) distance, gas bond lengths, and bond angles are calculated for the adsorbed gases, as shown in Table 2. Note that the N—O bond length 1 corresponds to the O—N bond distance for the gas oxygen atom interacting with the RE metal (RE-O—N—O); the N—O bond length 2 corresponds to the N—O bond associated with the “free” (not bound to the MOF) oxygen atom (RE-O—N—O)).

TABLE 2
Calculated binding energies, gas interaction distance, bond
lengths, and bond angles for YDOBDC with adsorbed NO₂ and H₂O
NO₂ binding

| | | | Bond |
|---|---|---|---|
| E_{binding} | O—RE | N—O bond length [Å] | angle |

| Metal | [kJ/mol] | [Å] | Bond 1 | Bond 2 | [°] |
|---|---|---|---|---|---|
| Y | −73.23 | 2.52 | 1.25 | 1.24 | 121.1 |
| Eu | −75.64 | 2.58 | 1.25 | 1.24 | 121.4 |
| Tb | −73.26 | 2.51 | 1.25 | 1.24 | 121.0 |
| Yb | −72.82 | 2.51 | 1.25 | 1.24 | 121.7 |

H₂O binding

| | | | Bond |
|---|---|---|---|
| E_{binding} | O—RE | O—H bond length [Å] | angle |

| Metal | [kJ/mol] | [Å] | Bond 1 | Bond 2 | [°] |
|---|---|---|---|---|---|
| Y | −101.59 | 2.43 | 0.99 | 0.98 | 110.6 |
| Eu | −100.91 | 2.50 | 0.99 | 0.99 | 110.4 |
| Tb | −101.90 | 2.43 | 0.98 | 1.00 | 110.3 |
| Yb | −106.45 | 2.38 | 0.99 | 0.98 | 111.1 |

In the minimum energy orientation, NO₂ has a vertical position, with one of the oxygen atoms interacting with the metal, while the other extends into the pore. This allows the pore oxygen in the NO₂ to interact with a neighboring DOBDC linker (shown as dashed line in FIG. 71B). The NO₂-DOBDC interaction impacts the binding energy while also modifying the NO₂ geometry. The calculated NO₂ bond lengths show a slight elongation when adsorbed to the metal sites, where the N—O bond lengths become 1.24-1.25 Å(Table 2). This indicates a slight structural change in the molecule compared to equilibrium bond lengths of 1.197 Å. See G. Herzberg, Molecular Spectra and Molecular Structure. Vol. 3: Electronic Spectra and Electronic Structure of Polyatomic Molecules, D. Van Nostrand Co., Inc. (Princeton, N.J.), 1966.

Similar calculations were carried out for H₂O binding to the individual metal centers. When the H₂O molecule binds with the metal center, the H—O—H bond angle expands by ˜6° from the initially calculated value of 104.7° (Table 2). See A. R. Hoy et al., J. Mol. Spectrosc. 74, 1(1979). Bond angle extension is a feature of structured water formed under high nanoconfinement, and as a comparison, hexagonal ice has a similar bond angle of 109°. See M. Chaplin, Biophys. Chem. 83, 211 (2000); and W. Kuhs et al., J. Phys. Chem. 87, 4312 (1983). The bond angle and size of the H₂O molecule may also be a factor in allowing the molecule to preferentially bind with the metal site compared with NO₂. With a much larger interatomic angle (H₂O=104° versus NO₂=134° in vacuum), NO₂ has a higher degree of steric hindrance in accessing the metal site, due to larger kinetic diameter than that of H₂O (3.4 Å vs 2.65 Å).

Overall, H₂O has a stronger binding energy than NO₂ with the metal sites in the RE-DOBDC MOF, which may be partially attributed to structural changes in the gas molecule when trapped within the confined MOF structure.

### Photoluminescence Properties of Exemplary MOF Compositions

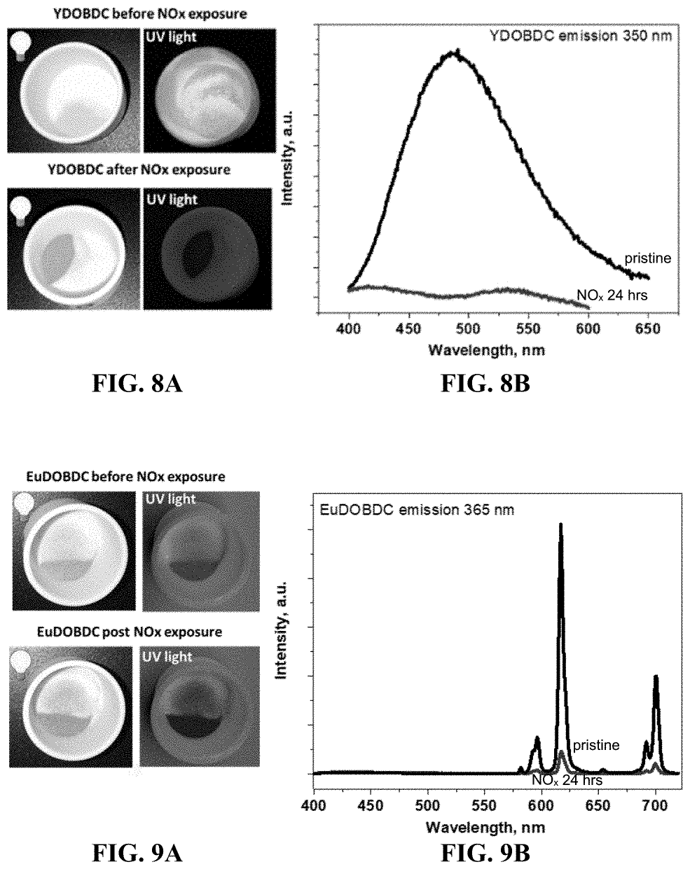

The photoluminescence (PL) properties before and after NOₓ exposures were also investigated. The intrinsic PL properties of this family of RE-MOFs has previously been examined in depth. See D. F. Sava Gallis et al., ACS Appl. Mater. Interfaces 9, 22268 (2017). Pristine desolvated compounds 1, 2, and 3 display broad, linker-based emission in the visible range, as shown in FIGS. 8A-8B. By comparison, compound 4 presents dominant metal (Eu)-based emission, as shown in FIGS. 9A-9B. Characteristic Eu³⁺ parity forbidden ⁵D-⁷F transitions at ˜590 nm (magnetic dipole transition ⁵D₀-⁷F₁), and electric dipole transitions at ˜616 nm (⁵D₀-⁷F₂), 650 nm (⁵D₀-⁷F₃), and 700 nm (⁵D₀-⁷F₄) are clearly identified in this system.

Interestingly, upon exposure to the NOₓ environment, almost immediately there are distinct changes in the physical appearance of the solids under visible light (from pale yellow to a vibrant brown-orange color), as shown in FIGS. 8A and 9A. Additionally, under UV light (350 nm), there is a drastic reduction in the emission intensity in all compounds, as shown in FIGS. 8B and 9B. This strong PL change in all the materials is optical evidence of the guest-framework interactions.

### Calculated Photophysical Properties of Exemplary MOF Compositions

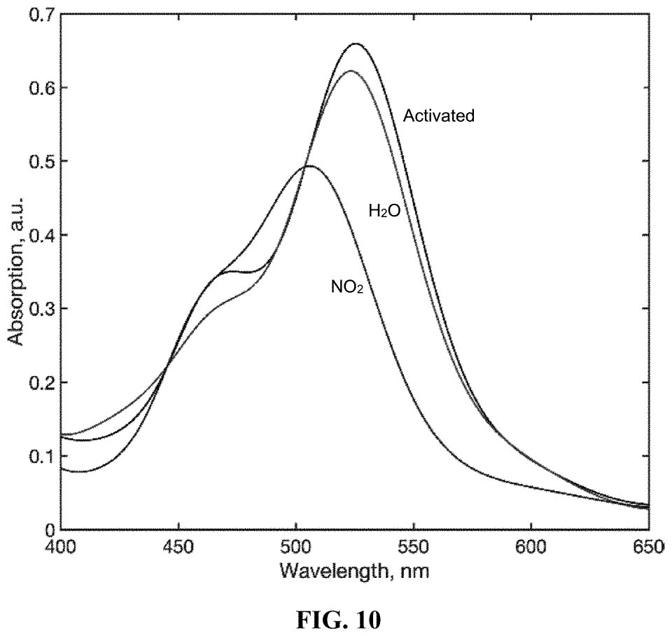

DFT simulations were next performed to identify features of the electronic structure of the MOF, which cause the reduction in photoemission intensity following NOₓ exposure. See D. F. Sava Gallis et al., ACS Appl. Mater. Interfaces 11, 43270 (2019); and D. J. Vogel et al., Phys. Chem. Chem. Phys. 21, 23085 (2019). Comparison of optical absorption spectra for pristine YDOBDC and with individual adsorbed H₂O and NO₂ gas species were calculated and indicated a distribution of transitions in the 400-650 nm energy range. This shows good qualitative agreement with the experimental PL characterization of broad linker emission, FIG. 8B (experimental) and FIG. 10 (calculation).

A comparison of the calculated absorption spectra for the activated YDOBDC, YDOBDC+H₂O and YDOBDC+NO₂ shows a distinct decrease in transition intensity with adsorption of both the H₂O and NO₂ molecules. This was investigated by examining the individual Kohn-Sham (KS) orbitals from the optimized ground state electronic structure. The ten strongest transitions within the H₂O and NO₂ adsorbed RE-MOF systems were calculated and produce individual adsorption spectra, as shown in Table 3. From them, the strongest transitions were calculated to be within the 400-650 nm energy range, consistent with the experimental optical adsorption range.

TABLE 3
Optical transitions in YDOBDC + H₂O and YDOBDC + NO₂, as
calculated by oscillator strength (fᵢⱼ), the energy of the transition (ωᵢⱼ),
and the corresponding transition label within the absorption spectra

| | |
|---|---|
| YDOBDC + H₂O | YDOBDC + NO₂ |

| Transition | | ωᵢⱼ | Transition | | ωᵢⱼ |
|---|---|---|---|---|---|
| label | fᵢⱼ | [nm] | label | fᵢⱼ | [nm] |
| A | 13.93 | 529 | A | 11.22 | 461 |
| B | 10.06 | 528 | B | 10.69 | 506 |
| C | 5.98 | 467 | C | 10.23 | 511 |
| D | 5.29 | 507 | D | 5.48 | 530 |
| E | 3.80 | 532 | E | 4.94 | 499 |
| F | 3.59 | 513 | F | 3.80 | 509 |
| G | 2.40 | 509 | G | 3.49 | 491 |
| | | | H | 3.01 | 464 |

Following adsorption of the NO₂ molecule into the MOF structure, energy states were introduced within the energy range of the transition states in the DOBDC ligand. The resulting change in the total electronic structure with NO₂ adsorption was visualized through comparison of the total density of state (DOS) for the activated and NO₂ systems. The adsorption of NO₂ also introduced a new unoccupied state at the valence band edge, creating an asymmetric electronic structure. From analysis of the separate spin states, the unoccupied state at the valence band edge was determined to be in the spin p (down) projection of the YDOBDC system. The new unoccupied state introduces new possible low energy transitions, which reduce the relative intensity of the transitions calculated within the same 400-650 nm range.

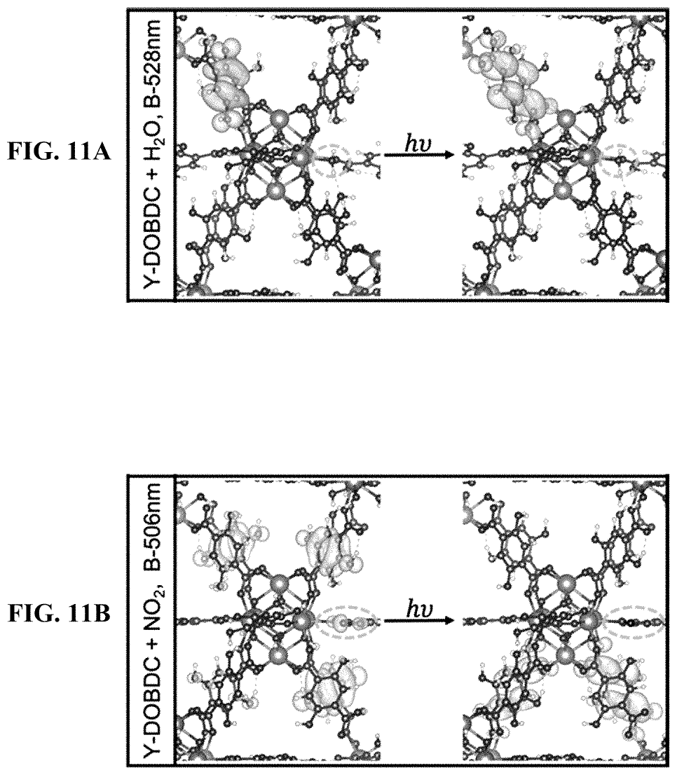

To further verify the calculated spectra, the partial charge densities of the KS orbitals were used to identify the material components involved in the highlighted photoluminescence transitions. Results show that, within the H₂O system, all transitions are localized on DOBDC ligands that are coordinated in bidentate fashion to two metal centers (see gray regions in FIG. 11A). These are the centers that are involved in the optical adsorption spectra of the molecule-MOF framework interaction. As an example, use of transition B (Table 3) for the H₂O system is shown in FIG. 11A.

Visualization of the KS orbitals participating in the NO₂ system shows that all highlighted transitions have electron density on the adsorbed NO₂ (see transition B, FIG. 11B). The absorption spectra have primary peaks of ˜520 nm and ˜502 nm for the H₂O and NO₂ systems, respectively. There is also a secondary shoulder near ˜467 nm and ˜461 nm, respectively, with the transitions calculated to be of similar character as the primary peak.

The calculated optical adsorption spectra for the RE-MOF is consistent with experimental photoluminescence adsorption spectra. Simulation results also indicate decreased optical adsorption with the addition of NO₂ to the MOF structure. This arises from both the addition of optical transitions with NO₂ adsorption, and the changes in the valence band edge of the DOS. Additionally, the optical transitions occur within the DOBDC ligand and the adsorbed NO₂ molecule, rather than with the RE metal center.

### Use of MOF Compositions as Advanced Absorbents of Acid Gases

The rare earth metal centered MOFs, RE-DOBDC, of the present invention show strong durability to the adsorption of humid NOₓ gases. Through a combination of computational modeling and materials characterization, an understanding of the structure-property relationship between the framework components and the preferential gas binding sites was elucidated. Pre- and post-humid NOₓ adsorption resulted in no structural change to the MOF structures, as determined by PXRD. Furthermore, contrary to the anticipated binding preferences, both calculations and materials characterization indicated that H₂O is preferentially binding to the metal center and that NOₓ is preferentially binding to the DOBDC ligands.

The interaction of both the H₂O and the NOₓ gas molecules with the MOF binding sites has a direct tie to understanding the framework stability. First principles DFT calculations indicate that H₂O has a stronger binding affinity to the metals, while the NOₓ is less strongly bound. These calculated binding energies are consistent across the rare earth elements used in differing analogs of RE-DOBDC MOFs. These data are supported by the materials characterization. New IR peaks post-NOₓ adsorption show a large variety of R—NO₂, organic nitrite R—ONO, and organic nitrate R—ONO₂ asymmetric and symmetric stretches are formed. Furthermore, it is important to point out that both the NO₂ and the H₂O molecules undergo subtle structural changes after MOF binding. This is likely caused by the nanoconfinement of the molecule and the interaction of the gas molecule with the framework ligands during binding.

Concurrently, obvious optical emission changes to the MOF pre- and post-humid NOₓ loading were another indication of preferential binding sites in the MOFs, independent of metal center. From the modeling, the emission spectra are derived primarily from the orbitals on the ligands. Perturbation of those orbitals by NOₓ binding resulted in a reduction of the emissions spectra. This was confirmed by experiments on the pre- and post-NOₓ loaded samples. In particular, all analogs displayed photoluminescence properties in their as-synthesized state; their emission was drastically reduced post-NOₓ loading. The ability of adsorbed NOₓ in this class of materials to nearly extinguish the emission from each of these MOFs highlights their feasibility to be incorporated into optical gas sensors.

### OTHER EMBODIMENTS

All publications, patents, and patent applications mentioned in this specification are incorporated herein by reference to the same extent as if each independent publication or patent application was specifically and individually indicated to be incorporated by reference.

While the invention has been described in connection with specific embodiments thereof, it will be understood that it is capable of further modifications and this application is intended to cover any variations, uses, or adaptations of the invention following, in general, the principles of the invention and including such departures from the present disclosure that come within known or customary practice within the art to which the invention pertains and may be applied to the essential features hereinbefore set forth, and follows in the scope of the claims.

Other embodiments are within the claims.

1. A method of detecting an acid gas, the method comprising:
providing a metal-organic framework composition comprising a plurality of metal clusters and a plurality of ligands coordinating with the plurality of metal clusters;
exposing the metal-organic framework composition to the acid gas; and
detecting a change in an optical emission spectrum of the metal-organic framework composition, as compared to before exposure to the acid gas.

2. The method of claim 1, wherein the detecting step comprises monitoring the optical emission spectrum while exciting the metal-organic framework composition with an ultraviolet light before and after exposure to the acid gas.

3. The method of claim 1, wherein the detecting step comprises exciting the metal-organic framework composition with an ultraviolet light and monitoring a decrease in an emission intensity at a wavelength within a visible spectrum, as compared to before exposure to the acid gas.

4. The method of claim 3, wherein the ultraviolet light has a wavelength of from about 320 to about 400 nm, and wherein the visible spectrum has a range of from about 400 nm to about 650 nm.

5. The method of claim 1, wherein the acid gas comprises nitrogen oxide, sulfur oxide, hydrogen sulfide, carbon dioxide, or mixtures thereof.

6. The method of claim 1, wherein at least one of the plurality of metal clusters comprises a hexanuclear cluster.

7. The method of claim 6, wherein the hexanuclear cluster comprises Zr, Eu, Nd, Yb, Y, Tb, La, Ce, Pr, Sm, Gd, Dy, Ho, Er, Tm, or Lu.

8. The method of claim 1, wherein the plurality of metal clusters comprises a first metal ion and a second metal ion that is different than the first metal ion.

9. The method of claim 8, wherein the plurality of metal clusters comprises a first metal ion having a first coordination geometry and a second metal ion having a second coordination geometry that is different than the first coordinate geometry.

10. The method of claim 1, wherein the metal-organic framework composition comprises a plurality of monodentate ligands and/or a plurality of bidentate ligands.

11. The method of claim 10, wherein at least one of the plurality of ligands comprises a structure of L¹-R^{L}-L², wherein each of L¹ and L² is, independently, a reactive group, and wherein R^{L} is a linker.

12. The method of claim 11, wherein R^{L} comprises an optionally substituted aryl or an optionally substituted heteroaryl.

13. The method of claim 12, wherein R^{L} comprises an aryl substituted with one or more of a hydroxyl, optionally substituted alkyl, haloalkyl, hydroxyalkyl, optionally substituted alkoxy, optionally substituted cycloalkyl, optionally substituted cycloalkoxy, optionally substituted aryl, optionally substituted aryloxy, halo, carboxyl, azido, cyano, nitro, amino, aminoalkyl, or carboxyaldehyde.

14. The method of claim 11, wherein each of L¹ and L² comprises, independently, carboxyl, heterocyclyl, hydroxyl, an anion thereof, a salt thereof, or an ester thereof.

15. The method of claim 1, wherein the plurality of metal clusters and plurality of ligands form a periodic framework.

16. The method of claim 1, wherein at least one of the plurality of ligands comprises a linear dicarboxylic acid.

17. The method of claim 1, wherein the metal-organic framework composition comprises RE-DOBDC, wherein RE is a rare earth element and DOBDC is 2,5-dihydroxyterephthalic acid.

18. The method of claim 17, wherein the rare earth element comprises Y, Yb, Tb, Eu, or combinations thereof.

19. The method of claim 1, wherein the metal-organic framework composition comprises UiO-66-DOBDC, UiO-66, UiO-67, NU-1000, MOF-808, or PCN-777.

20. A method of capturing an acid gas, the method comprising:
providing a metal-organic framework composition comprising a plurality of metal clusters and a plurality of ligands coordinating with the plurality of metal clusters, wherein at least one of the plurality of metal clusters comprises a hexanuclear cluster; and
exposing the metal-organic framework composition to the acid gas.

21. The method of claim 20, further comprising:
detecting a change in an optical emission spectrum of the metal-organic framework composition, as compared to before exposure to the acid gas, thereby confirming capture of the acid gas.

22. The method of claim 20, wherein the acid gas comprises nitrogen oxide, sulfur oxide, hydrogen sulfide, carbon dioxide, or mixtures thereof.

23. The method of claim 20, wherein the hexanuclear cluster comprises Zr, Eu, Nd, Yb, Y, Tb, La, Ce, Pr, Sm, Gd, Dy, Ho, Er, Tm, or Lu.

24. The method of claim 20, wherein the plurality of metal clusters comprises a first metal ion and a second metal ion that is different than the first metal ion.

25. The method of claim 24, wherein the plurality of metal clusters comprises a first metal ion having a first coordination geometry and a second metal ion having a second coordination geometry that is different than the first coordinate geometry.

26. The method of claim 20, wherein the metal-organic framework composition comprises a plurality of monodentate ligands and/or a plurality of bidentate ligands.

27. The method of claim 26, wherein at least one of the plurality of ligands comprises a structure of L¹-R^{L}-L², wherein each of L¹ and L² is, independently, a reactive group, and wherein R^{L} is a linker.

28. The method of claim 27, wherein R^{L} comprises an optionally substituted aryl or an optionally substituted heteroaryl.

29. The method of claim 28, wherein R^{L} comprises an aryl substituted with one or more of a hydroxyl, optionally substituted alkyl, haloalkyl, hydroxyalkyl, optionally substituted alkoxy, optionally substituted cycloalkyl, optionally substituted cycloalkoxy, optionally substituted aryl, optionally substituted aryloxy, halo, carboxyl, azido, cyano, nitro, amino, aminoalkyl, or carboxyaldehyde.

30. The method of claim 27, wherein each of L¹ and L² comprises, independently, carboxyl, heterocyclyl, hydroxyl, an anion thereof, a salt thereof, or an ester thereof.

31. The method of claim 20, wherein the plurality of metal clusters and plurality of ligands form a periodic framework.

32. The method of claim 20, wherein at least one of the plurality of ligands comprises a linear dicarboxylic acid.

33. The method of claim 20, wherein the metal-organic framework composition comprises a RE-DOBDC, wherein RE is a rare earth element and DOBDC is 2,5-dihydroxyterephthalic acid.

34. The method of claim 33, wherein the rare earth element comprises Y, Yb, Tb, Eu, or combinations thereof.

35. The method of claim 20, wherein the metal-organic framework composition comprises UiO-66-DOBDC, UiO-66, UiO-67, NU-1000, MOF-808, or PCN-777.
